(19) United States
(12) Patent Application Publication
(10) Pub. No.: US 2024/0279190 A1
(43) Pub. Date: Aug. 22, 2024

(54) COMPOUNDS FOR MODULATING PLATELET-TYPE 12-(S)-LIPOXYGENASE AND METHODS OF USE FOR SAME

(71) Applicant: The Regents of The University of California, Oakland, CA (US)

(72) Inventors: THEODORE HOLMAN, Santa Cruz, CA (US); SCOTT LOKEY, Santa Cruz, CA (US); MATTHEW JACOBSON, San Francisco, CA (US); CHAKRAPANI KALYANARAMAN, San Francisco, CA (US); JERRY NADLER, Valhalla, NY (US); WAN-CHEN TSAI, Santa Cruz, CA (US)

(21) Appl. No.: 18/289,214

(22) PCT Filed: May 6, 2022
(86) PCT No.: PCT/US2022/028119

§ 371 Date: Nov. 1, 2023

Related U.S. Application Data
(60) Provisional application No. 63/185,958, filed on May 7, 2021.
(60) Provisional application No. 63/196,115, filed on Jun. 2, 2021.

(51) Int. Cl. C07D 277/84; C07D 513/04

(52) U.S. Cl. CPC C07D 277/84; C07D 513/04

(57) ABSTRACT

Compounds for inhibiting human platelet-type 12-(S)-lipoxygenase (12-LOX) are provided. Compounds according to certain embodiments modulate platelet activation and hemostasis. In some embodiments, compounds described herein modulate platelet activation mediated by immune receptor, FcγRIIa. Methods for modulating platelet reactivity (e.g., following vascular insult or injury) with the compounds are also described. Methods for treating or preventing a platelet-type 12-(S)-lipoxygenase (12-LOX)-mediated disease are also provided. Compositions for practicing the subject methods are also described.

### CROSS-REFERENCE TO RELATED APPLICATIONS

Pursuant to 35 U.S.C. § 119 (e), this application claims priority to the filing date of U.S. Provisional Patent Application Ser. No. 63/185,958 filed May 7, 2021 and U.S. Provisional Patent Application Ser. No. 63/196,115 filed Jun. 2, 2021; the disclosure of which application is incorporated herein by reference in their entirety.

### INTRODUCTION

Human Platelet-type 12-(S)-lipoxygenase (12-LOX) is a non-heme iron-containing oxygenase that catalyzes the regio- and stereo-specific addition of molecular oxygen to polyunsaturated fatty acids (PUFA). 12-LOX belongs to a family of enzymes that also include 5-LOX and 15-LOX, which oxygenate arachidonic acid (AA) at their corresponding carbon positions. The hydroperoxyeicosatetraenoic acid (HPETE) product is subsequently reduced by cellular peroxidases to form the hydroxyeicosatetraenoic acid (HETE), which in the case of 12-LOX is 12-(S)-HETE.

Although 12-LOX expression is predominantly restricted to platelets (˜14,000 molecules per platelet), it is also expressed in some hematopoietic and solid tumors. To date, 12-LOX is the only LOX isoform identified to be present in platelets, and its activity is part of a number of platelet functions, including granule secretion, platelet aggregation, and normal adhesion through specific agonist-mediated pathways, such as collagen and the thrombin receptor, PAR4. Normal platelet activation plays a central role in the regulation of hemostasis, but uncontrolled activation can lead to pathologic thrombotic events, such as ischemic coronary heart disease.

### SUMMARY

Compounds for inhibiting human platelet-type 12-(S)-lipoxygenase (12-LOX) are provided. Compounds according to certain embodiments modulate platelet activation and hemostasis. In some embodiments, compounds described herein modulate platelet activation mediated by immune receptor, FcγRIIa. Methods for modulating platelet reactivity (e.g., following vascular insult or injury) with the compounds are also described. Methods for treating or preventing a platelet-type 12-(S)-lipoxygenase (12-LOX)-mediated disease are also provided. In certain embodiments, platelet reactivity is modulated while minimizing increased risk of bleeding as compared to other forms of antiplatelet therapy. Compositions for practicing the subject methods are also described.

In some embodiments, compounds of interest include a compound of formula (I):

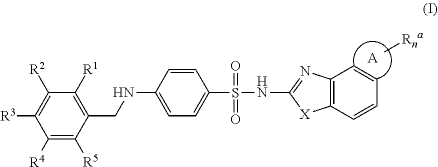

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

In some embodiments, R⁸ is hydroxy. In some embodiments, R² is alkoxy. In some instances, R² is a C₁-C₁₂ alkoxy. In some instances, R² is selected from methoxy, ethoxy, propoxy, butoxy, isobutoxy and tert-butoxy. In certain instances, R² is methoxy. In some instances, R³ is hydrogen. In some instances, R⁴ is hydrogen. In some instances, R⁵ is hydrogen. In some instances, each of R³, R⁴ and R⁵ are hydrogen.

In some embodiments, X is oxygen. In other embodiments, X is S.

In some embodiments, A is a six-membered ring. In some instances, A is a six-membered heterocyclic ring. In some instances, A is a six-membered aryl ring. In some instances, A is a six-membered heteroaryl ring. In some embodiments, A is a six-membered ring and the compound is of formula (IA):

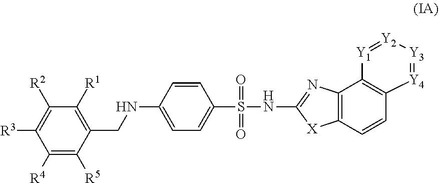

where Y₁, Y₂, Y₃ and Y₄ are each independently C or N. In some embodiments, Y₁, Y₂, Y₃ and Y₄ are each C. In some embodiments, Y₃ is N and Y₁, Y₂, and Y₄ are each C. In some embodiments, Y₁ and Y₄ are N; and Y₂ and Y₃ are C. In some embodiments, A is unsubstituted. In some embodiments, A is monosubstituted. In other embodiments, A is di-substituted. In other embodiments, A is tri-substituted. In some instances, n is 1 or 2 and each R^{a} is independently selected from:

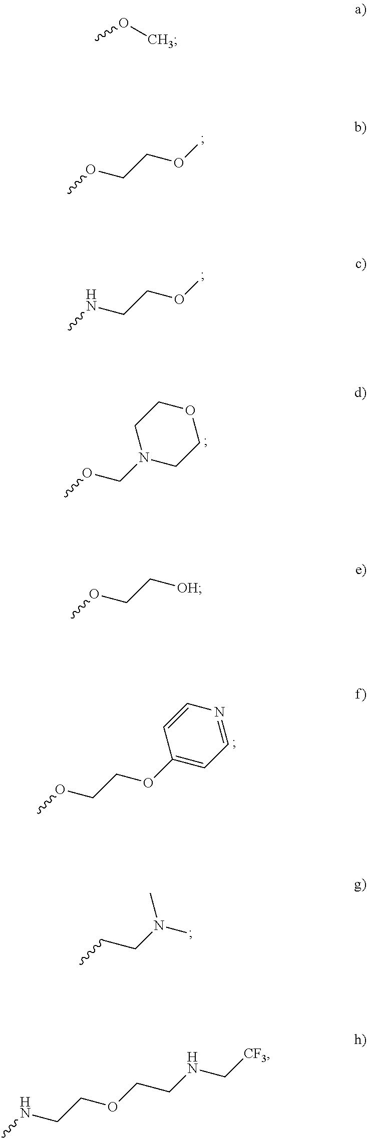

where [symbol] represents the A-R^{a} bond.

In some embodiments, A is a five-membered ring. In some instances, A is a five-membered heterocyclic ring. In some instances, A is a five-membered heteroaryl ring. In some embodiments, A is a five-membered ring and the compound is of formula (IB):

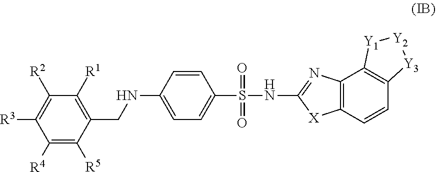

where Y₁, Y₂, Y₃ and Y₄ are each independently C, N or O.

In some embodiments, Y₁, Y₂ and Y₃ are each C. In some embodiments, Y₂ is C and Y₁, and Y₃ are each O. In some embodiments, Y₁ is N; and Y₂ and Y₃ are C. In some embodiments, Y₃ is N; and Y₁ and Y₂ are C. In some embodiments, Y₁ is O; Y₂ is C and Y₃ is N. In some embodiments, Y₁ is N; Y₂ is C and Y₃ is O. In some embodiments, A is unsubstituted. In some embodiments, A is monosubstituted. In other embodiments, A is di-substituted. In other embodiments, A is tri-substituted. In some instances, n is 1 or 2 and each R^{a} is independently selected from:

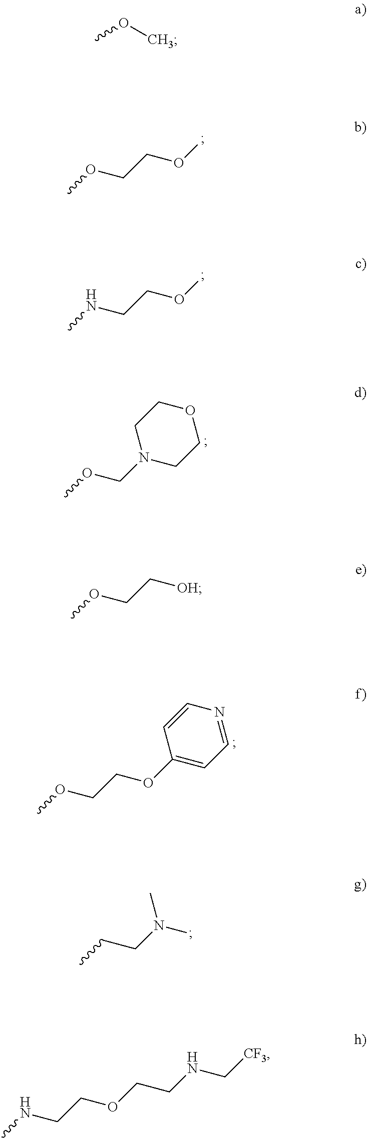

where [symbol] represents the A-R^{a} bond.

In certain embodiments, the compound is 4-((2-hydroxy-3-methoxybenzyl)amino)-N-(naphtho[1,2-d]thiazol-2-yl)benzenesulfonamide:

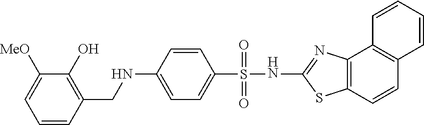 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.

Aspects of the disclosure also include methods for modulating or inhibiting platelet-type 12-(S)-lipoxygenase (12-LOX) by contacting a cell with an amount of the subject compounds or a pharmaceutically acceptable salt thereof. In some instances, the cell having platelet-type 12-(S)-lipoxygenase (12-LOX) is contacted with the compound in vitro. In other instances, the cell having platelet-type 12-(S)-lipoxygenase (12-LOX) is contacted with the compound in vivo. In some instances, methods include contacting one or more of the compounds described herein with cells having platelet-type 12-(S)-lipoxygenase (12-LOX) in a manner sufficient to reduce 12-hydroxyeicosatetraenoic acid (12-HETE) in cells (e.g., human cells). In some embodiments, methods include modulating platelet activation and hemostasis. In some embodiments, methods include modulating platelet activation mediated by immune receptor, FcγRIIa. In some embodiments, methods include modulating PAR4-AP (protease-activated receptor 4) induced platelet aggregation. In some embodiments, methods include modulating PAR4-AP induced calcium mobilization.

In some embodiments, methods include treating or preventing a 12-lipoxygenase mediated disease, such as where the disease includes production of 12-hydroperoxyeicosatetraenoic acid (12(S)-HPETE) or 12-hydroxyeicosatetraenoic acid (12(S)-HETE). In some embodiments, methods include treating or preventing an immune-mediated thrombocytopenia or thrombosis disorder. In some embodiments, methods include administering one or more of the compounds described herein to a subject diagnosed with one or more of type I diabetes, type II diabetes, diabetic kidney disease, diabetic nerve disease, cardiovascular disease, non-alcoholic steatohepatitis, platelet hemostasis, heparin-induced thrombocytopenia, thrombosis, Alzheimer's disease and cancer.

### BRIEF DESCRIPTION OF THE FIGURES

FIG. 1 depicts predicted binding mode of ML355 with wt12-LOX. Carbon atoms of ML355 are shown in turquoise, whereas carbon atoms of the protein are shown in gray color. Nitrogen, oxygen, hydrogen and sulfur atoms are shown in blue, red, white and yellow colors respectively. Residues interacting with ML355 and the metal ion are also shown. Residues that were mutated in this study are shown in ball-and-stick representation and labelled, whereas other residues are shown in stick representation. A hydroxide ion interacting with Fe³⁺ is also shown in ball-and-stick representation. Fe³⁺ ion is shown as an orange sphere. Hydrogen bonds are shown in cyan color.

FIG. 2 depicts the correlation of pIC₅₀ values of ML355 against human 12-LOX mutants to the docking scores.

FIG. 3 depicts the relative cavity shapes and sizes of MHL355 binding in the active site of (A) wt12-LOX (B) L407A and (C) L407G.

FIG. 4 depicts the relative cavity sizes of MHL355 binding in the active site of (A) wt12-LOX and (B) A417I/V418M (Sloane determinants)

FIG. 5 depicts the predicted binding mode of Compound LOX-12-001 with wt12-LOX. Residues that interact with the inhibitor are shown. Residues that mutated in the present study are shown in ball-and-stick representation and they are labelled. Carbon atoms of compound LOX-12-001 are shown in turquoise color, whereas carbon atoms of wt12-LOX are shown in gray color. Oxygen, nitrogen, hydrogen and sulfur atoms are shown in red, blue, white and yellow colors respectively. Fe³⁺ ion is shown as orange sphere. Hydroxide ion is shown in ball-and-stick representation.

FIG. 6A depicts relative cavity sizes of compound LOX-12-001 binding in the active site of (A) wt12-LOX and (B) A417I/V418M (Sloane determinants). FIG. 6B depicts the predicted binding mode of MHL355 in the active site of wt12-LOX showing the closest distances of benzothiazole ring to aromatic side chains of F352 and F414.

FIG. 7 depicts the predicted binding mode of MHL355 in the active site of wt12-LOX interacting with H596 (A) and H596L (B).

FIG. 8 depicts batch incubation of human islets treated with PIC with or without the h12-LOX inhibitor, compound LOX-12-001. GSIS was compared between human islets that were untreated (Ctl), pretreated with PIC, pretreated with PIC plus compound LOX-12-001 (1 μM) or pretreated with compound LOX-12-001 (1 μM) for 24 hours. Each condition from a single donor was performed in triplicate, and data from three donors were combined (n=8 to 9). **P<0.01;***P<0.005 by Two-way Annova's variance Sidak's multiple comparison test.

FIG. 9 depicts interactions of the binding site of wt12-LOX with compound LOX-12-001.

### DEFINITIONS

The following terms have the following meaning unless otherwise indicated. Any undefined terms have their art recognized meanings.

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, succinyl, and malonyl, and the like.

The term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ═N—N═, —N═N—, —N═N—NR³⁹R⁴⁰, —PR⁴¹—, —P(O)₂—, —POR⁴²—, —O—P(O)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, 3-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O⁻, ═O, —OR⁶⁰, —SR⁶⁰, —S⁻, ═S, —NR⁶⁰R⁶¹, ═NR⁶⁰, —CF₃, —CN, —OCN, —SCN, —NO, —NO₂, ═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —C(S)OR⁶⁰, —NR⁶²C(O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶⁴ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶⁴ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶, —S⁻, ═S, —NR⁶⁰R⁶¹, ═NR⁶⁰, —CF₃, —CN, —OCN, —SCN, —NO, —NO₂, ═N₂, —N₃, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O⁻, —NR⁶²C(O)NR⁶⁰R⁶¹. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include -M, —R⁶⁰, ═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)OR⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

“Pharmaceutically acceptable carrier” refers to a diluent, adjuvant, excipient or vehicle with, or in which a compound is administered.

### DETAILED DESCRIPTION

Compounds for inhibiting human platelet-type 12-(S)-lipoxygenase (12-LOX) are provided. Compounds according to certain embodiments modulate platelet activation and hemostasis. In some embodiments, compounds described herein modulate platelet activation mediated by immune receptor, FcγRIIa. Methods for modulating platelet reactivity (e.g., following vascular insult or injury) with the compounds are also described. Methods for treating or preventing a platelet-type 12-(S)-lipoxygenase (12-LOX)-mediated disease are also provided. Compositions for practicing the subject methods are also described.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the compounds and methods have or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

Reference will now be made in detail to various embodiments. It will be understood that the invention is not limited to these embodiments. To the contrary, it is intended to cover alternatives, modifications, and equivalents as may be included within the spirit and scope of the allowed claims.

### Compounds for Modulating Platelet-Type 12-(S)-Lipoxygenase

In some embodiments, compounds of the present disclosure include a compound of formula (I):

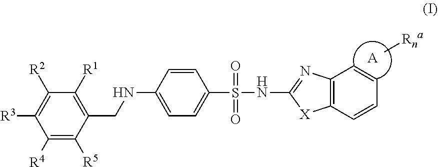

where R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

In embodiments, “salts” of the compounds of the present disclosure may include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

The term “solvate” as used herein refers to a complex or aggregate formed by one or more molecules of a solute, e.g. a compound of Formula (I) or a salt thereof, and one or more molecules of a solvent. Such solvates may be crystalline solids having a substantially fixed molar ratio of solute and solvent. Representative solvents include by way of example, water, methanol, ethanol, isopropanol, acetic acid, and the like. When the solvent is water, the solvate formed is a hydrate.

In some instances, R¹ is hydroxy. In some instances, R¹ is hydrogen. In some instances, R² is alkoxy. In some instances, R² is a C₁-C₁₂ alkoxy. In some instances, R² is selected from methoxy, ethoxy, propoxy, butoxy, isobutoxy and tert-butoxy. In certain instances, R² is methoxy. In some instances, R² is hydrogen. In some instances, R³ is hydrogen. In some instances, R⁴ is hydrogen. In some instances, R⁵ is hydrogen. In some instances, each of R³, R⁴ and R⁵ are hydrogen.

In some instances, X is O. In some instances, X is S.

In some instances, A is a six-membered ring. In some instances, A is a six-membered heterocyclic ring. In some instances, A is a six-membered aryl ring. In some instances, A is a six-membered heteroaryl ring.

In some instances, the compound is of formula (IA):

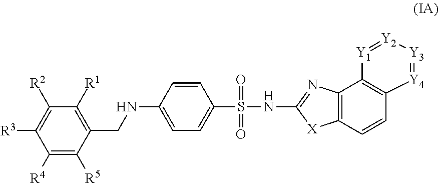

where Y₁, Y₂, Y₃ and Y₄ are each independently C or N.

In some instances, Y₁, Y₂, Y₃ and Y₄ are each C and the compound is of formula (IA1):

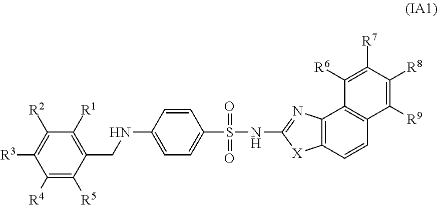

where R⁶, R⁷, R⁸ and R⁹ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

In some instances, R⁶ is hydrogen. In some instances, R⁷ is hydrogen. In some instances, R⁸ is hydrogen. In some instances, R⁹ is hydrogen. In some instances, each of R⁶, R⁷, R⁸ and R⁹ are hydrogen.

In some instances, R⁷ is selected from:

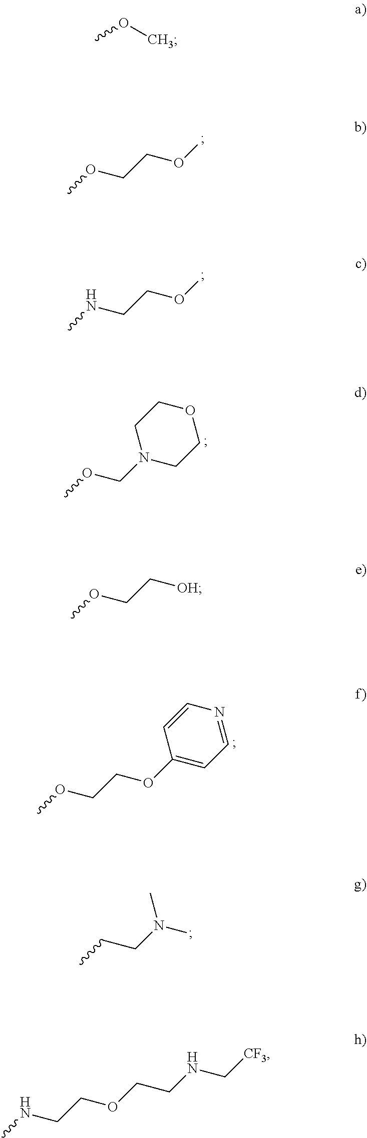

where [symbol] represents the A-R⁷ bond.

In some instances, R⁸ is selected from:

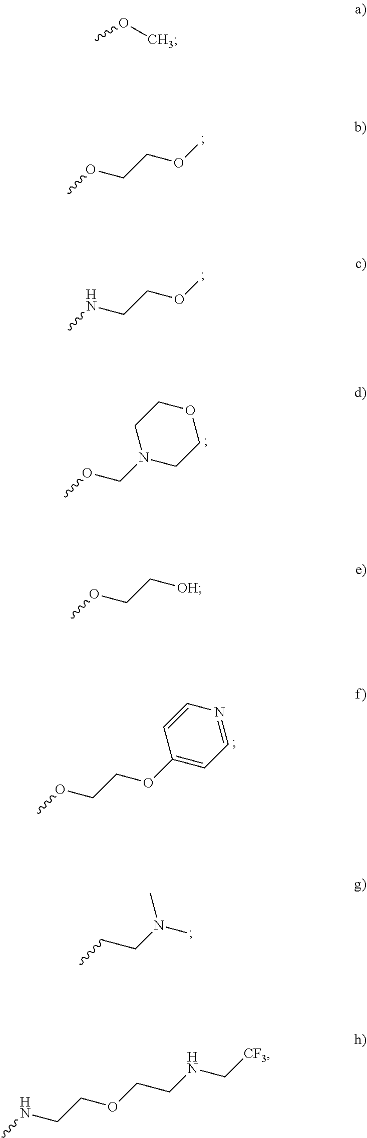

where [symbol] represents the A-R⁸ bond.

In some embodiments, R⁷ and R⁸ together with the carbon to which they are attached form a cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl. In some instances, R⁷ and R⁸ together with the carbon to which they are attached form a 6-membered ring. In certain instances, R⁷ and R⁸ together with the carbon to which they are attached form a heterocyclic 6-membered ring. In some instances, R⁷ and R⁸ together with the carbon to which they are attached form a 5-membered ring. In certain instances, R⁷ and R⁸ together with the carbon to which they are attached form a heterocyclic 5-membered ring. In certain instances, R⁷ and R⁸ together with the carbon to which they are attached form a heterocyclic 5-membered ring and the compound is of formula (IA1a):

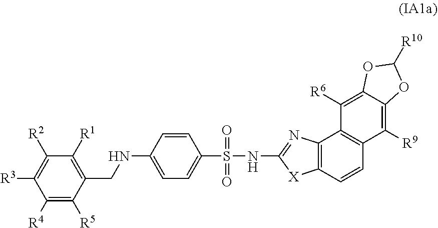

where R¹⁰ is hydrogen, hydroxy, alkoxy, amine, cyano, thiol, halogen, alkyl or selected from:

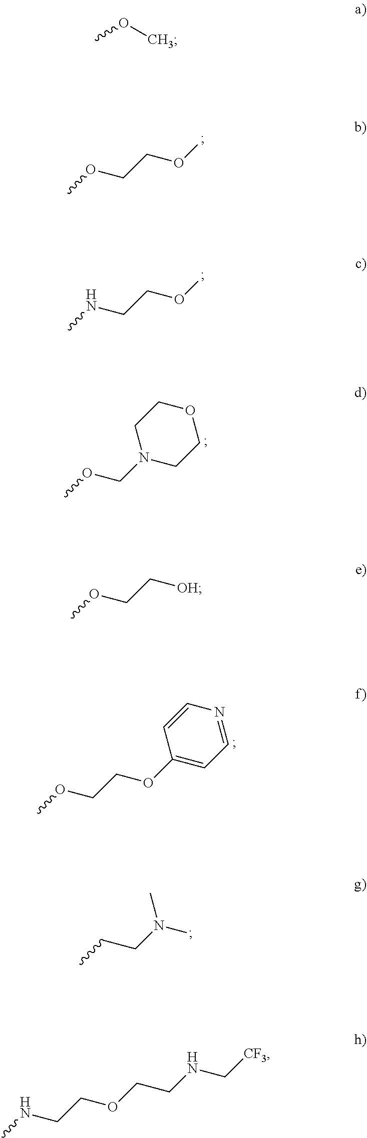

where [symbol] represents the C—R¹⁰ bond.

In some instances, Y₃ is N and Y₁, Y₂, and Y₄ are each C and the compound is of formula (IA2):

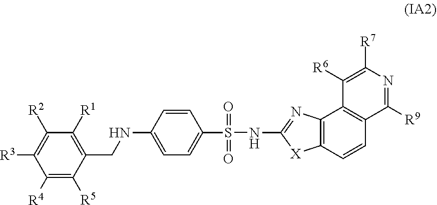

where R⁶, R⁷, R⁸ and R⁹ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

In some instances, R⁶ is hydrogen. In some instances, R⁷ is hydrogen. In some instances, R⁹ is hydrogen. In some instances, each of R⁶, R⁷ and R⁹ are hydrogen.

In some instances, R⁷ is selected from:

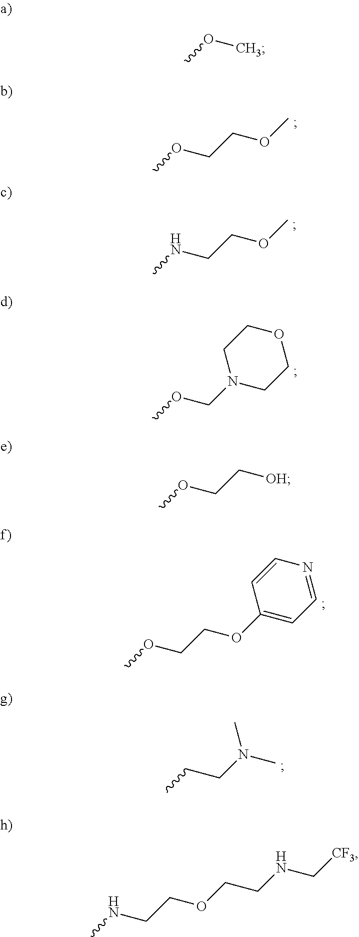

where [symbol] represents the A-R⁷ bond.

In some instances, Y₁ and Y₄ are N and Y₂ and Y₃ are each C and the compound is of formula (IA3):

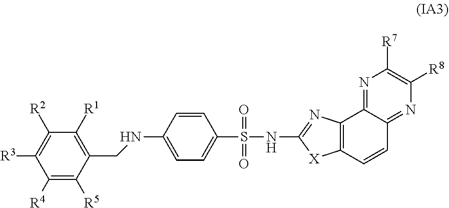

where R⁷ and R⁸ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl. In some embodiments, R⁷ and R⁸ together with the carbon to which they are attached form a cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl. In some instances, R⁷ and R⁸ together with the carbon to which they are attached form a 6-membered ring. In certain instances, R⁷ and R⁸ together with the carbon to which they are attached form a heterocyclic 6-membered ring. In some instances, R⁷ and R⁸ together with the carbon to which they are attached form a 5-membered ring. In certain instances, R⁷ and R⁸ together with the carbon to which they are attached form a heterocyclic 5-membered ring.

In some instances, R⁷ is hydrogen. In some instances, R⁸ is hydrogen. In some instances, In some instances, R⁷ and R⁸ are hydrogen. In some instances, R⁷ and R⁸ are independently selected from:

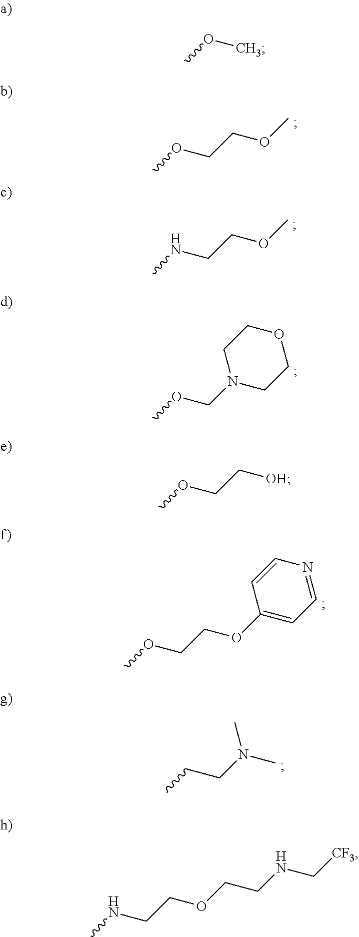

where [symbol] represents the A-R⁷ bond or the A-R⁸ bond.

In some embodiments, compounds of interest include those shown in Table A, which are not to be construed as limitative.

TABLE A

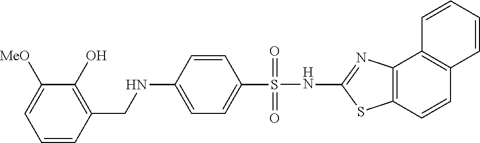 [SMILES: COc1cccc(CNc2ccc(S(=O)(=O)Nc3nc4c(ccc5ccccc54)s3)cc2)c1O]

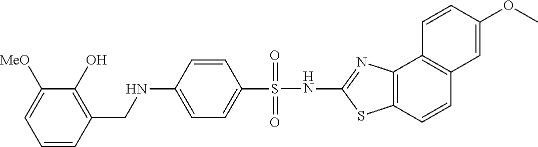 [SMILES: COc1ccc2c(ccc3sc(NS(=O)(=O)c4ccc(NCc5cccc(OC)c5O)cc4)nc32)c1]

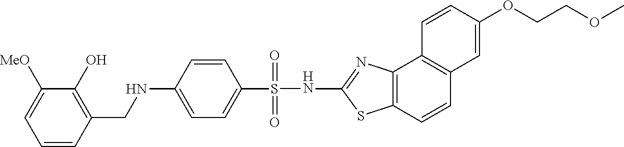 [SMILES: COCCOc1ccc2c(ccc3sc(NS(=O)(=O)c4ccc(NCc5cccc(OC)c5O)cc4)nc32)c1]

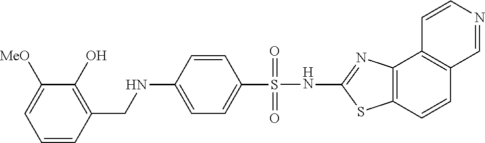 [SMILES: COc1cccc(CNc2ccc(S(=O)(=O)Nc3nc4c(ccc5cnccc54)s3)cc2)c1O]

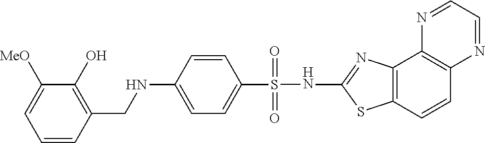 [SMILES: COc1cccc(CNc2ccc(S(=O)(=O)Nc3nc4c(ccc5nccnc54)s3)cc2)c1O]

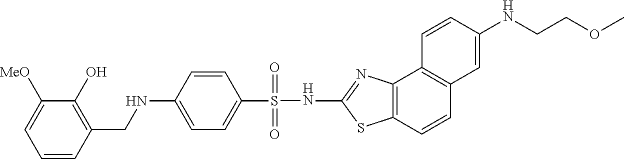 [SMILES: COCCNc1ccc2c(ccc3sc(NS(=O)(=O)c4ccc(NCc5cccc(OC)c5O)cc4)nc32)c1]

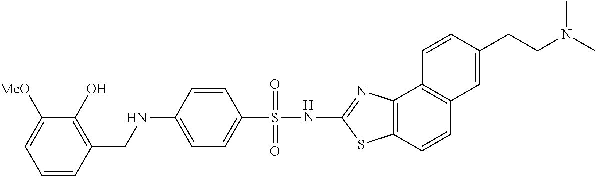 [SMILES: COc1cccc(CNc2ccc(S(=O)(=O)Nc3nc4c(ccc5cc(CCN(C)C)ccc54)s3)cc2)c1O]

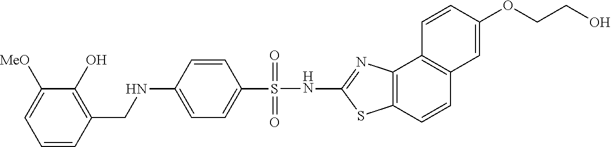 [SMILES: COc1cccc(CNc2ccc(S(=O)(=O)Nc3nc4c(ccc5cc(OCCO)ccc54)s3)cc2)c1O]

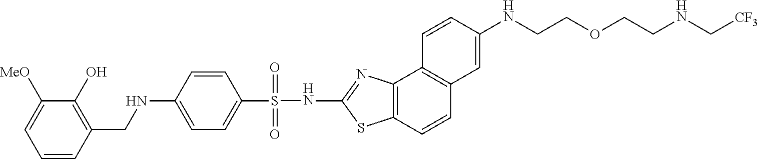 [SMILES: COc1cccc(CNc2ccc(S(=O)(=O)Nc3nc4c(ccc5cc(NCCOCCNCC(F)(F)F)ccc54)s3)cc2)c1O]

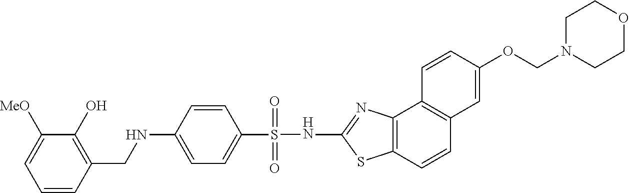 [SMILES: COc1cccc(CNc2ccc(S(=O)(=O)Nc3nc4c(ccc5cc(OCN6CCOCC6)ccc54)s3)cc2)c1O]

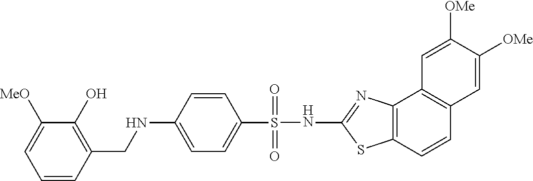 [SMILES: COc1cc2ccc3sc(NS(=O)(=O)c4ccc(NCc5cccc(OC)c5O)cc4)nc3c2cc1OC]

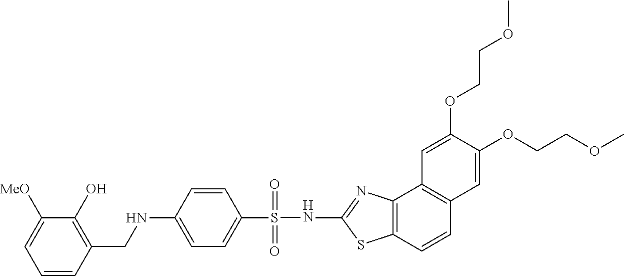 [SMILES: COCCOc1cc2ccc3sc(NS(=O)(=O)c4ccc(NCc5cccc(OC)c5O)cc4)nc3c2cc1OCCOC]

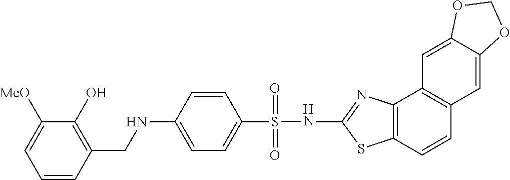 [SMILES: COc1cccc(CNc2ccc(S(=O)(=O)Nc3nc4c(ccc5cc6c(cc54)OCO6)s3)cc2)c1O]

In some instances, A is a five-membered ring. In some instances, A is a five-membered heterocyclic ring. In some instances, A is a five-membered aryl ring. In some instances, A is a five-membered heteroaryl ring.

In some instances, the compound is of formula (Ia):

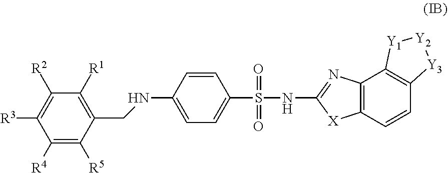

where Y₁, Y₂ and Y₃ are each independently C or N. In some embodiments, Y₁, Y₂ and Y₃ are each C. In some embodiments, Y₂ is C and Y₁, and Y₃ are each O. In some embodiments, Y₁ is N; and Y₂ and Y₃ are C. In some embodiments, Y₃ is N; and Y₁ and Y₂ are C. In some embodiments, Y₁ is O; Y₂ is C and Y₃ is N. In some embodiments, Y₁ is N; Y₂ is C and Y₃ is O.

In some instances, Y₁, Y₂ and Y₃ are each C and the compound is of formula (IB1):

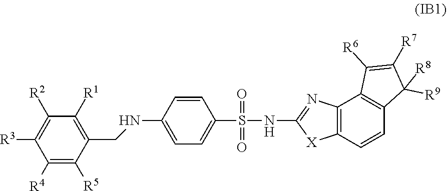

where R⁶, R⁷, R⁸ and R⁹ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl.

In some instances, R⁶ is hydrogen. In some instances, R⁷ is hydrogen. In some instances, R⁸ is hydrogen. In some instances, R⁹ is hydrogen. In some instances, each of R⁶, R⁷, R⁸ and R⁹ are hydrogen.

In some instances, Y₂ is C and Y₁, and Y₃ are each O and the compound is of formula (IB2):

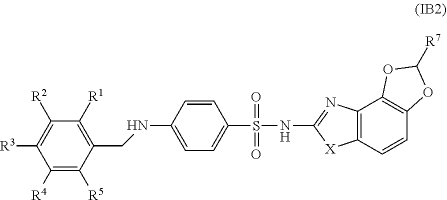

where R⁷ is hydrogen, hydroxy, alkoxy, amine, cyano, thiol, halogen, alkyl or selected from:

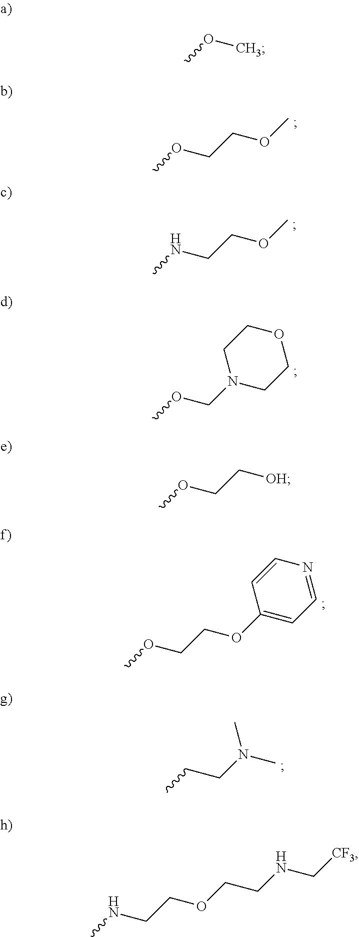

where [symbol] represents the C—R⁷ bond.

In some instances, Y₁ is N; and Y₂ and Y₃ are C and the compound is of formula (IB3):

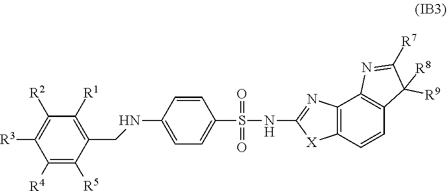

where R⁷, R⁸ and R⁹ are independently hydrogen, hydroxy, alkoxy, amine, cyano, thiol, halogen, alkyl or selected from:

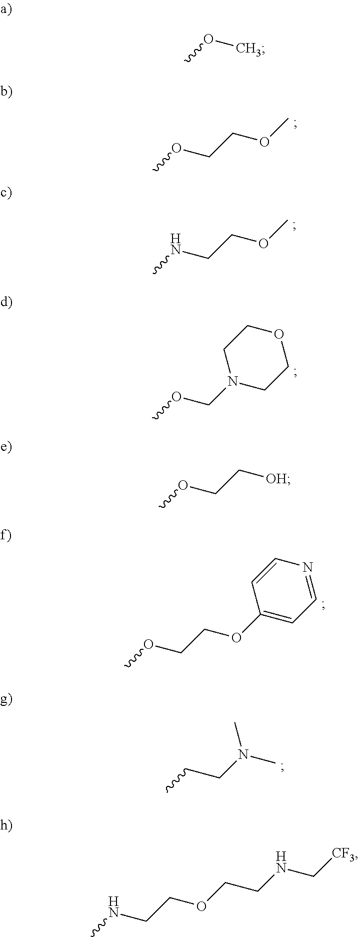

where [symbol] represents the C—R bond.

In some instances, R⁷ is hydrogen. In some instances, R⁸ is hydrogen. In some instances, R⁹ is hydrogen. In some instances, each of R⁷, R⁸ and R⁹ are hydrogen.

In some instances, Y₁ and Y₂ are C; and Y₃ is C and the compound is of formula (IB4):

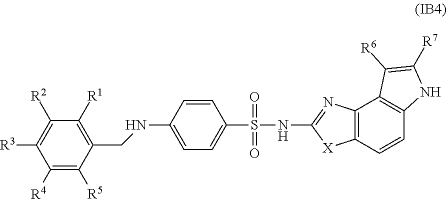

where R⁶ and R⁷ are independently hydrogen, hydroxy, alkoxy, amine, cyano, thiol, halogen, alkyl or selected from:

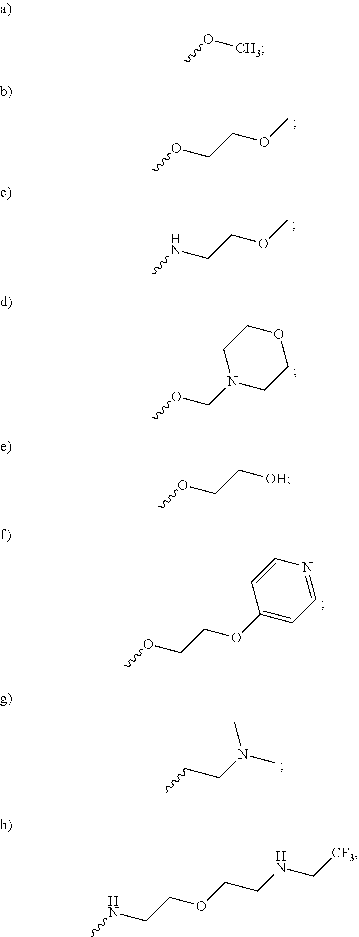

where [symbol] represents the C—R bond.

In some instances, R⁶ is hydrogen. In some instances, R⁷ is hydrogen. In some instances, each of R⁶ and R⁷ are hydrogen.

In some instances, Y₁ is N; Y₂ is C and Y₃ is O and the compound is of formula (IB5):

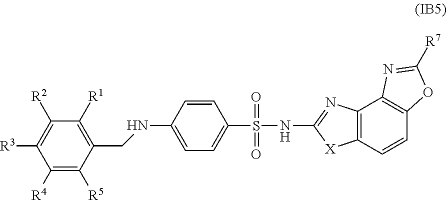

where R⁷ is hydrogen, hydroxy, alkoxy, amine, cyano, thiol, halogen, alkyl or selected from:

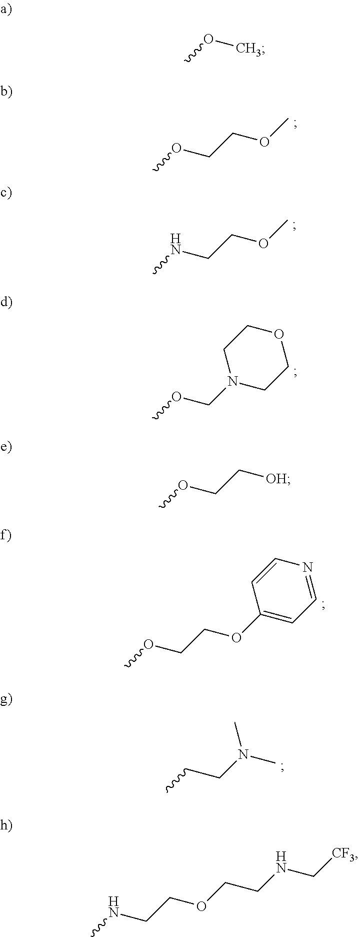

where [symbol] represents the C—R⁷ bond.

In some instances, Y₁ is O; Y₂ is C and Y₃ is N and the compound is of formula (IB6):

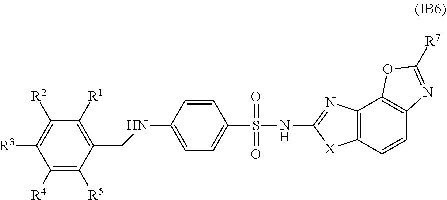

where R⁷ is hydrogen, hydroxy, alkoxy, amine, cyano, thiol, halogen, alkyl or selected from:

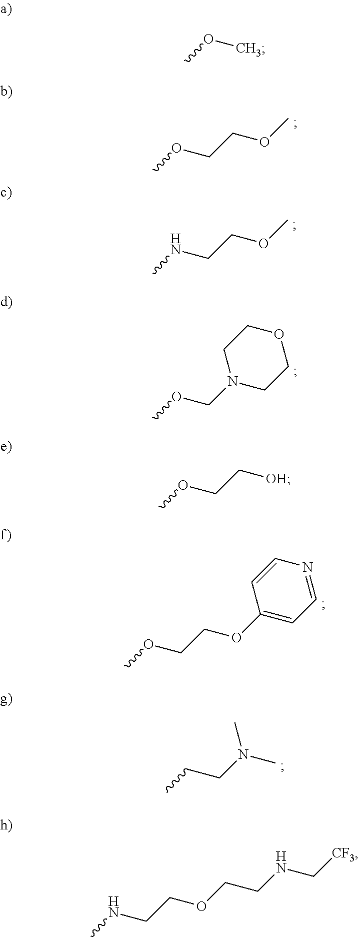

where [symbol] represents the C—R⁷ bond.

In some embodiments, compounds of interest include those shown in Table B, which are not to be construed as limitative.

TABLE B

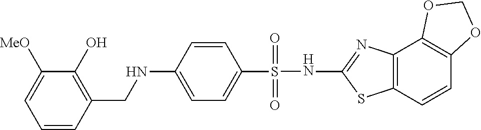 [SMILES: COc1cccc(CNc2ccc(S(=O)(=O)Nc3nc4c5c(ccc4s3)OCO5)cc2)c1O]

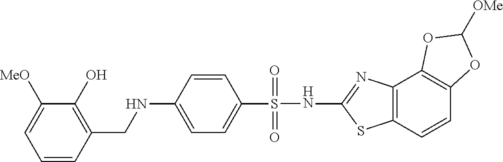 [SMILES: COc1cccc(CNc2ccc(S(=O)(=O)Nc3nc4c5c(ccc4s3)OC(OC)O5)cc2)c1O]

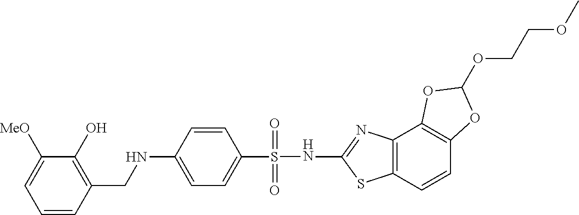 [SMILES: COCCOC1Oc2ccc3sc(NS(=O)(=O)c4ccc(NCc5cccc(OC)c5O)cc4)nc3c2O1]

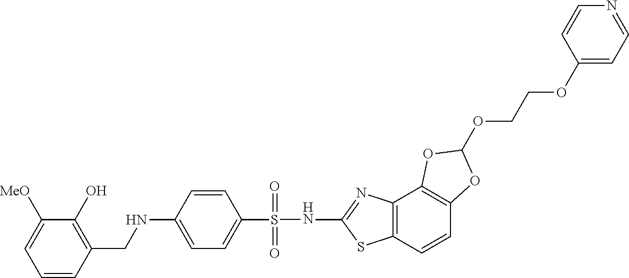 [SMILES: COc1cccc(CNc2ccc(S(=O)(=O)Nc3nc4c5c(ccc4s3)OC(OCCOc3ccncc3)O5)cc2)c1O]

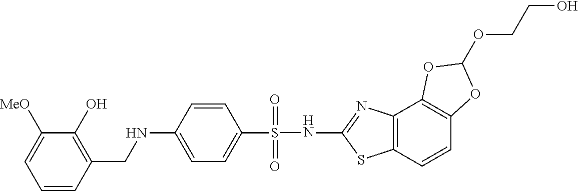 [SMILES: COc1cccc(CNc2ccc(S(=O)(=O)Nc3nc4c5c(ccc4s3)OC(OCCO)O5)cc2)c1O]

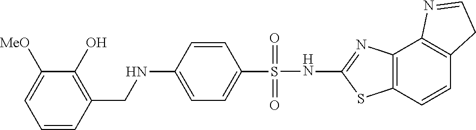 [SMILES: COc1cccc(CNc2ccc(S(=O)(=O)Nc3nc4c5c(ccc4s3)CC=N5)cc2)c1O]

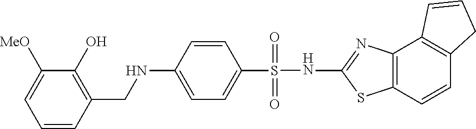 [SMILES: COc1cccc(CNc2ccc(S(=O)(=O)Nc3nc4c5c(ccc4s3)CC=C5)cc2)c1O]

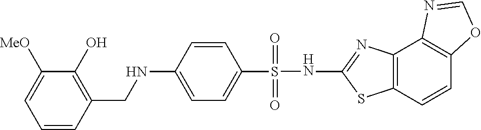 [SMILES: COc1cccc(CNc2ccc(S(=O)(=O)Nc3nc4c(ccc5ocnc54)s3)cc2)c1O]

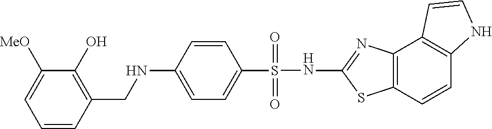 [SMILES: COc1cccc(CNc2ccc(S(=O)(=O)Nc3nc4c(ccc5[nH]ccc54)s3)cc2)c1O]

In certain embodiments, the compound is 4-((2-hydroxy-3-methoxybenzyl)amino)-N-(naphtho[1,2-d]thiazol-2-yl)benzenesulfonamide:

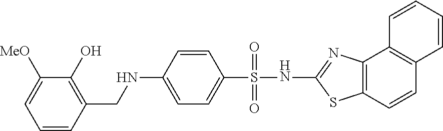 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.

Aspects of the present disclosure also include compositions having a pharmaceutically acceptable carrier and one or more of the compounds described above. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7th ed., Lippincott, Williams, & Wilkins; and Handbook of Pharmaceutical Excipients (2000) A. H. Kibbe et al., eds., 3rd ed. Amer. Pharmaceutical Assoc. For example, the one or more excipients may include sucrose, starch, mannitol, sorbitol, lactose, glucose, cellulose, talc, calcium phosphate or calcium carbonate, a binder (e.g., cellulose, methylcellulose, hydroxymethylcellulose, polypropylpyrrolidone, polyvinylpyrrolidone, gelatin, gum arabic, poly(ethylene glycol), sucrose or starch), a disintegrator (e.g., starch, carboxymethylcellulose, hydroxypropyl starch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinylpyrrolidone or aluminum stearate), a dispersing agent (e.g., hydroxypropylmethylcellulose), a diluent (e.g., water), and base wax (e.g., cocoa butter, white petrolatum or polyethylene glycol).

The compounds may be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. In certain embodiments, the conjugate compounds are formulated for injection. For example, compositions of interest may be formulated for intravenous or intraperitoneal administration.

In pharmaceutical dosage forms, the compounds may be administered in the form of its pharmaceutically acceptable salts, or it may also be used alone or in appropriate association, as well as in combination, with other pharmaceutically active compounds. The following methods and excipients are merely exemplary and are in no way limiting.

In some embodiments, compositions of interest include an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. In some instances, compositions of interest further include a preservative. Suitable preservatives include, but are not limited to, a benzyl alcohol, phenol, chlorobutanol, benzalkonium chloride, and the like. In many cases, the composition is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, compositions include other additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Where the composition is formulated for injection, the compounds may be formulated by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Although the dosage used in treating a subject will vary depending on the clinical goals to be achieved, a suitable dosage range of the compound is one which provides up to about 0.0001 mg to about 5000 mg, e.g., from about 1 mg to about 25 mg, from about 25 mg to about 50 mg, from about 50 mg to about 100 mg, from about 100 mg to about 200 mg, from about 200 mg to about 250 mg, from about 250 mg to about 500 mg, from about 500 mg to about 1000 mg, or from about 1000 mg to about 5000 mg of an active agent, which can be administered in a single dose. Those of skill will readily appreciate that dose levels can vary as a function of the specific compound, the severity of the symptoms and the susceptibility of the subject to side effects.

In some embodiments, a single dose of the compound is administered. In other embodiments, multiple doses of the compound are administered. Where multiple doses are administered over a period of time, the compound may be administered, e.g., twice daily (qid), daily (qd), every other day (qod), every third day, three times per week (tiw), or twice per week (biw) over a period of time. For example, the compound may be administered qid, qd, qod, tiw, or biw over a period of from one day to about 2 years or more. For example, the compound may be administered at any of the aforementioned frequencies for one week, two weeks, one month, two months, six months, one year, or two years, or more, depending on various factors.

Dose units of the present disclosure can be made using manufacturing methods available in the art and can be of a variety of forms suitable for injection (including topical, intracisternal, intrathecal, intravenous, intramuscular, subcutaneous and dermal) administration, for example as a solution, suspension, solution, lyophilate or emulsion. The dose unit can contain components conventional in pharmaceutical preparations, e.g. one or more carriers, binders, lubricants, excipients (e.g., to impart controlled release characteristics), pH modifiers, coloring agents or further active agents.

Dose units can comprise components in any relative amounts. For example, dose units can be from about 0.1% to 99% by weight of active ingredients (i.e., compounds described herein) per total weight of dose unit. In some embodiments, dose units can be from 10% to 50%, from 20% to 40%, or about 30% by weight of active ingredients per total weight dose unit.

### Methods for Modulating Platelet-Type 12(S)-Lipoxygenase (12-LOX)

As summarized above, aspects of the present disclosure also modulating or inhibiting platelet-type 12-(S)-lipoxygenase (12-LOX). In certain instances, the 12-LOX is human platelet-type 12-(S)-lipoxygenase. In some embodiments, methods include contacting a cell having platelet-type 12-(S)-lipoxygenase (12-LOX) with one or more of the compounds described herein in vitro. In other embodiments, methods include contacting a cell having platelet-type 12-(S)-lipoxygenase (12-LOX) with one or more of the compounds described herein in vivo (e.g., by administering to a subject as described in greater detail below). In still other embodiments a cell having platelet-type 12-(S)-lipoxygenase (12-LOX) is contacted ex vivo. In some embodiments, methods include decreasing or reducing 12-LOX activity, such as reducing 12-LOX activity by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more.

In some embodiments, the subject methods include modulating platelet activation. In certain embodiments, methods including reducing or inhibiting platelet activation, such as where platelet activation is reduced by 1% or more, such as by 5% or more, such as by 10% or more, such as by 15% or more, such as by 20% or more, such as by 25% or more, such as by 30% or more, such as by 35% or more, such as by 40% or more, such as by 45% or more, such as by 50% or more, such as by 60% or more, such as by 70% or more, such as by 80% or more, such as by 90% or more, such as by 95% or more, such as by 97% or more, such as by 99% or more and including by 99.9% or more. In certain embodiments, methods include modulating platelet activation mediated by immune receptor, FcγRIIa. In certain instances, methods include reducing platelet activation mediated by immune receptor, FcγRIIa. In certain embodiments, methods include contacting one or more of the compounds described herein with cells having platelet-type 12-(S)-lipoxygenase (12-LOX) in a manner sufficient to reduce 12-hydroxyeicosatetraenoic acid (12-HETE) in cells (e.g., human cells).

In some instances, methods include treating or preventing a 12-lipoxygenase mediated disease. The term “treat” or “treatment” of any condition, refers, in certain embodiments, to ameliorating the condition (i.e., arresting or reducing the development of the condition). In certain embodiments “treating” or “treatment” refers to ameliorating at least one physical parameter, which may not be discernible by the patient. In certain embodiments, “treating” or “treatment” refers to inhibiting the condition, either physically, (e.g., stabilization of a discernible symptom), physiologically, (e.g., stabilization of a physical parameter), or both. In certain embodiments, “treating” or “treatment” refers to delaying the onset of the condition. The term “therapeutically effective amount” is used herein to refer to the amount of a compound that, when administered to a patient for preventing or treating a condition is sufficient to effect such treatment. The “therapeutically effective amount” will vary depending on the compound, the condition and its severity and the age, weight, etc., of the patient.

In practicing the subject methods, a therapeutically effective amount of one or more of the compounds disclosed herein is administered to a subject sufficient to treat or prevent the 12-lipoxygenase mediated disease. In embodiments, the term “subject” is meant the person or organism to which the compound is administered. As such, subjects of the present disclosure may include but are not limited to mammals, e.g., humans and other primates, such as chimpanzees and other apes and monkey species, dogs, rabbits, cats and other domesticated pets; and the like, where in certain embodiments the subject are humans. The term “subject” is also meant to include a person or organism of any age, weight or other physical characteristic, where the subjects may be an adult, a child, an infant or a newborn.

In some instances, the 12-lipoxygenase mediated disease is an immune-mediated thrombocytopenia or thrombosis, such as for example heparin-induced thrombocytopenia, anti-phospholipid syndrome, sepsis syndrome, thrombosis associated with therapeutic or diagnostic monoclonal antibodies and thrombotic thrombocytopenic purpura. In some embodiments, methods include treating a subject for one or more of type I diabetes, type II diabetes, diabetic kidney disease, diabetic nerve disease, cardiovascular disease, non-alcoholic steatohepatitis, platelet hemostasis, heparin-induced thrombocytopenia, thrombosis, Alzheimer's disease and cancer.

Compounds as described herein may be administered to a subject by any convenient protocol, including, but not limited, to intraperitoneally, topically, orally, sublingually, parenterally, intravenously, vaginally, rectally as well as by transdermal protocols. In certain embodiments, the subject compounds are administered by intravenous injection. In certain embodiments, the subject compounds are administered by intraperitoneal injection.

Depending on the condition being treated, the amount of compound administered to the subject may vary, such as ranging from about 0.0001 mg/day to about 10,000 mg/day, such as from about 0.001 mg/day to about 9000 mg/day, such as from 0.01 mg/day to about 8000 mg/day, such as from about 0.1 mg/day to about 7000 mg/day, such as from about 1 mg/day to about 6000 mg/day, including from about 5 mg/day to about 5000 mg/day. Each dosage of the compound or pharmaceutically acceptable salt administered to the subject may vary ranging from about 1 mg/kg to about 1000 mg/kg, such as from about 2 mg/kg to about 900 mg/kg, such as from about 3 mg/kg to about 800 mg/kg, such as from about 4 mg/kg to about 700 mg/kg, such as from 5 mg/kg to about 600 mg/kg, such as from 6 mg/kg to about 500 mg/kg, such as from 7 mg/kg to about 400 mg/kg, such as from about 8 mg/kg to about 300 mg/kg, such as from about 9 mg/kg to about 200 mg/kg and including from about 10 mg/kg to about 100 mg/kg.

In certain embodiments, protocols may include multiple dosage intervals. By “multiple dosage intervals” is meant that two or more dosages of the compound is administered to the subject in a sequential manner. In practicing methods of the present disclosure, treatment regimens may include two or more dosage intervals, such as three or more dosage intervals, such as four or more dosage intervals, such as five or more dosage intervals, including ten or more dosage intervals. The duration between dosage intervals in a multiple dosage interval treatment protocol may vary, depending on the physiology of the subject or by the treatment protocol as determined by a health care professional. For example, the duration between dosage intervals in a multiple dosage treatment protocol may be predetermined and follow at regular intervals. As such, the time between dosage intervals may vary and may be 1 day or longer, such as 2 days or longer, such as 4 days or longer, such as 6 days or longer, such as 8 days or longer, such as 12 days or longer, such as 16 days or longer and including 24 days or longer. In certain embodiments, multiple dosage interval protocols provide for a time between dosage intervals of 1 week or longer, such as 2 weeks or longer, such as 3 weeks or longer, such as 4 weeks or longer, such as 5 weeks or longer, including 6 weeks or longer.

The cycles of drug administration may be repeated for 1, 2, 3, 4, 5, 6, 7, 8 or more than 8 dosage cycles, for a total period of 6 months or 1 year or 2 years or 3 years or 4 years or more. In certain embodiments, one or more of the subject compounds are administered for the rest of the subject's lifetime.

In certain embodiments, compounds of the present disclosure can be administered prior to, concurrent with, or subsequent to other therapeutic agents for treating the same or an unrelated condition. If provided at the same time as another therapeutic agent, the present compounds may be administered in the same or in a different composition. Thus, the compounds of interest and other therapeutic agents can be administered to the subject by way of concurrent therapy. By “concurrent therapy” is intended administration to a subject such that the therapeutic effect of the combination of the substances is caused in the subject undergoing therapy. For example, concurrent therapy may be achieved by administering the compounds of the present disclosure with a pharmaceutical composition having at least one other agent, such as an anti-inflammatory agent, immunosuppressant, steroid, analgesic, anesthetic, antihypertensive, chemotherapeutic, among other types of therapeutics, which in combination make up a therapeutically effective dose, according to a particular dosing regimen. Administration of the separate pharmaceutical compositions can be performed simultaneously or at different times (i.e., sequentially, in either order, on the same day, or on different days), so long as the therapeutic effect of the combination of these substances is caused in the subject undergoing therapy.

Where the compounds of the present disclosure is administered concurrently with a second therapeutic agent to treat the same condition (e.g., a chemotherapeutic, an anti-viral drug, etc.) the weight ratio of the subject compound to second therapeutic agent may range from 1:2 and 1:2.5; 1:2.5 and 1:3; 1:3 and 1:3.5 1:3.5 and 1:4; 1:4 and 1:4.5; 1:4.5 and 1:5; 1:5 and 1:10; and 1:10 and 1:25 or a range thereof. For example, the weight ratio of the subject compound to second therapeutic agent may range between 1:1 and 1:5; 1:5 and 1:10; 1:10 and 1:15; or 1:15 and 1:25. Alternatively, the weight ratio of the second therapeutic agent to the subject compound ranges between 2:1 and 2.5:1; 2.5:1 and 3:1; 3:1 and 3.5:1; 3.5:1 and 4:1; 4:1 and 4.5:1; 4.5:1 and 5:1; 5:1 and 10:1; and 10:1 and 25:1 or a range thereof. For example, the ratio of the second therapeutic agent the subject compound may range between 1:1 and 5:1; 5:1 and 10:1; 10:1 and 15:1; or 15:1 and 25:1.

Aspects, including embodiments, of the subject matter described herein may be beneficial alone or in combination, with one or more other aspects or embodiments. Without limiting the description, certain non-limiting aspects of the disclosure numbered 1-34 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A compound of formula I:

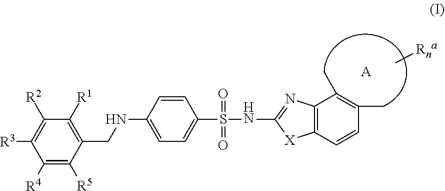

wherein R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.
2. The compound according to 1, wherein each of R³, R⁴ and R⁵ are hydrogen.
3. The compound according to any one of 1-2, wherein R¹ is hydroxy.
4. The compound according to any one of 1-3, wherein R² is alkoxy.
5. The compound according to 4, wherein R² is methoxy.
6. The compound according to any one of 1-5, wherein X is S.
7. The compound according to any one of 1-6, wherein A is a six-membered ring.
8. The compound according to 7, wherein A is a six-membered heteroaryl ring.
9. The compound according to any one of 7-8, wherein the compound is of formula Ia:

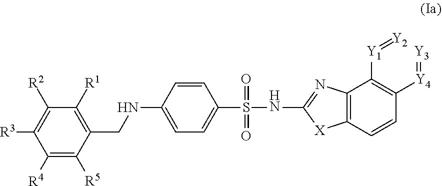

wherein Y₁, Y₂, Y₃ and Y₄ are each independently C or N.
10. The compound according to 9, wherein Y₁, Y₂, Y₃ and Y₄ are each C.
11. The compound according to 9, wherein:
   Y₃ is N; and
   Y₁, Y₂, and Y₄ are each C.
12. The compound according to 9, wherein:
   Y₁ and Y₄ are N; and
   Y₂ and Y₃ are C.
13. The compound according to any one of 7-12, wherein A is unsubstituted.
14. The compound according to any one of 7-12, wherein:
   n is 1 or 2; and
   each R^{a} is independently selected from:

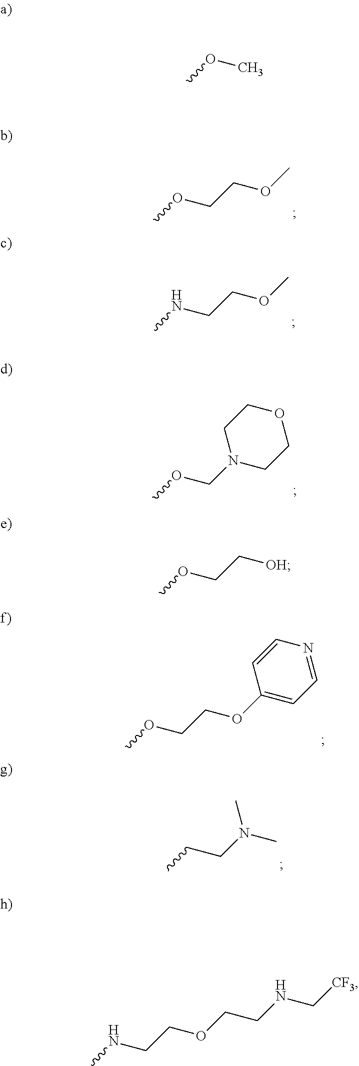

wherein [symbol] represents the A-R^{a} bond.
15. The compound according to any one of 7-14, wherein the compound is selected from:

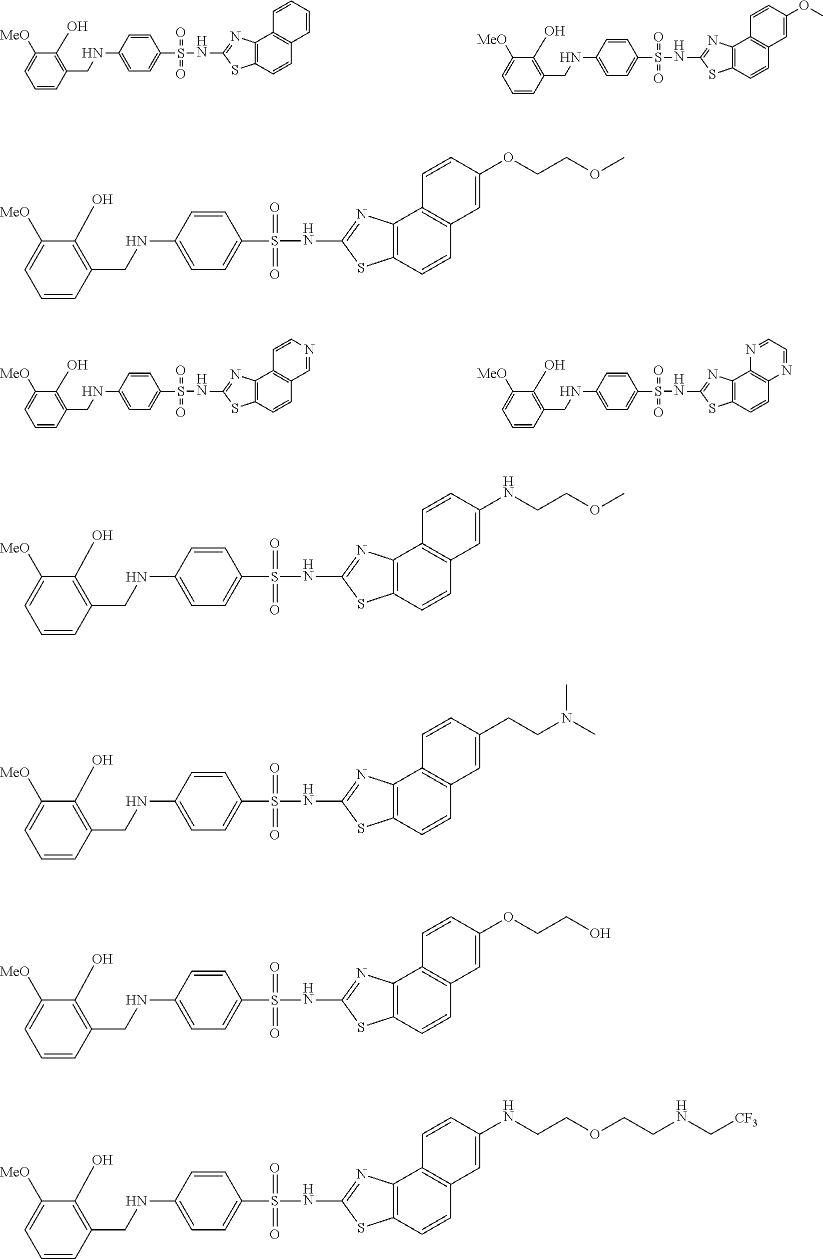

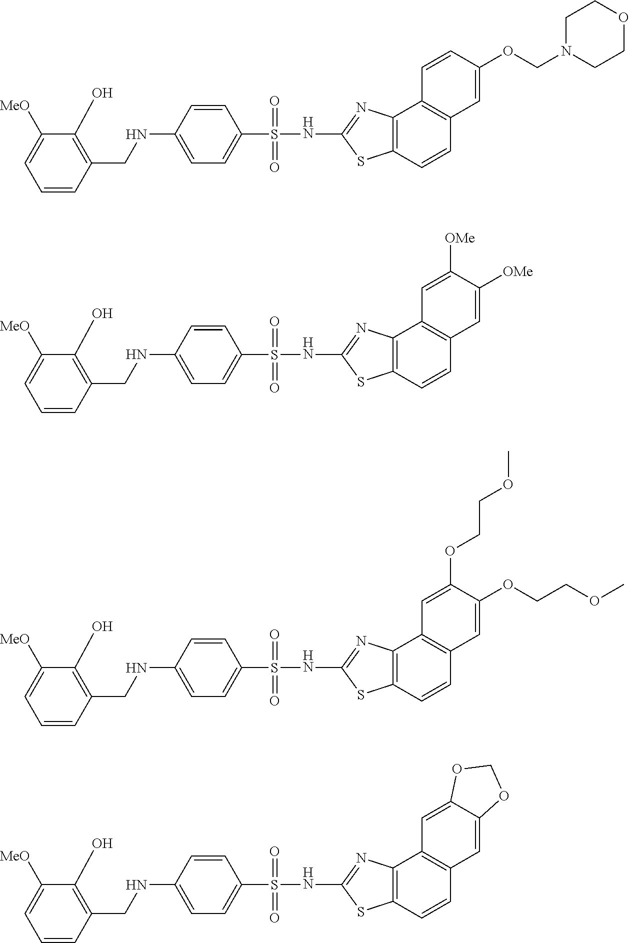 [SMILES: COc1cccc(CNc2ccc(S(=O)(=O)Nc3nc4c(ccc5cc(OCN6CCOCC6)ccc54)s3)cc2)c1O ; COc1cc2ccc3sc(NS(=O)(=O)c4ccc(NCc5cccc(OC)c5O)cc4)nc3c2cc1OC ; COCCOc1cc2ccc3sc(NS(=O)(=O)c4ccc(NCc5cccc(OC)c5O)cc4)nc3c2cc1OCCOC ; COc1cccc(CNc2ccc(S(=O)(=O)Nc3nc4c(ccc5cc6c(cc54)OCO6)s3)cc2)c1O]

16. The compound according to any one of 1-6, wherein A is a five-membered ring.
17. The compound according to 16, wherein A is a five-membered heterocyclic ring.
18. The compound according to 16, wherein A is a five-membered heteroaryl ring.
19. The compound according to any one of 16-18, wherein the compound is of formula Ib:

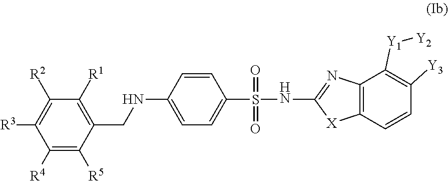

wherein Y₁, Y₂, Y₃ and Y₄ are each independently C, N or O.
20. The compound according to 19, wherein Y₁, Y₂ and Y₃ are each C.
21. The compound according to 20, wherein:
   Y₂ is C; and
   Y₁ and Y₃ are O.
22. The compound according to 20, wherein:
   Y₁ is N; and
   Y₂ and Y₃ are C.
23. The compound according to any one of 16-22, wherein A is unsubstituted.
24. The compound according to any one of 16-22, wherein:
   n is 1 or 2; and
   each R^{a} is independently selected from:

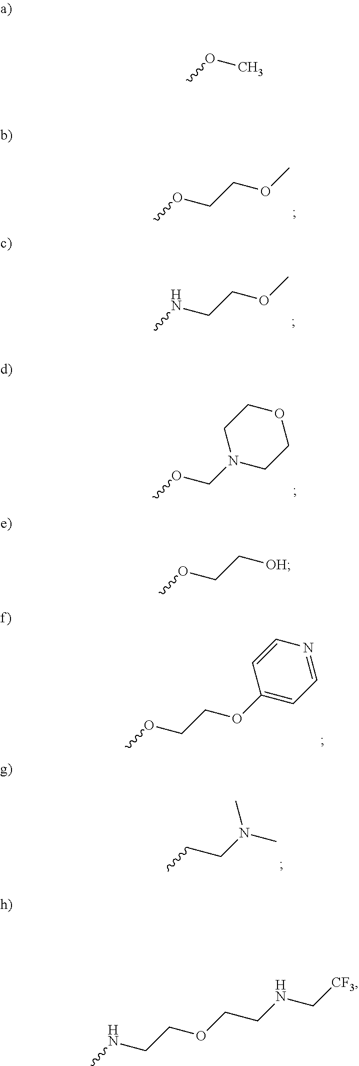

wherein [symbol] represents the A-R^{a} bond.
25. The compound according to any one of 16-24, wherein the compound is selected from:

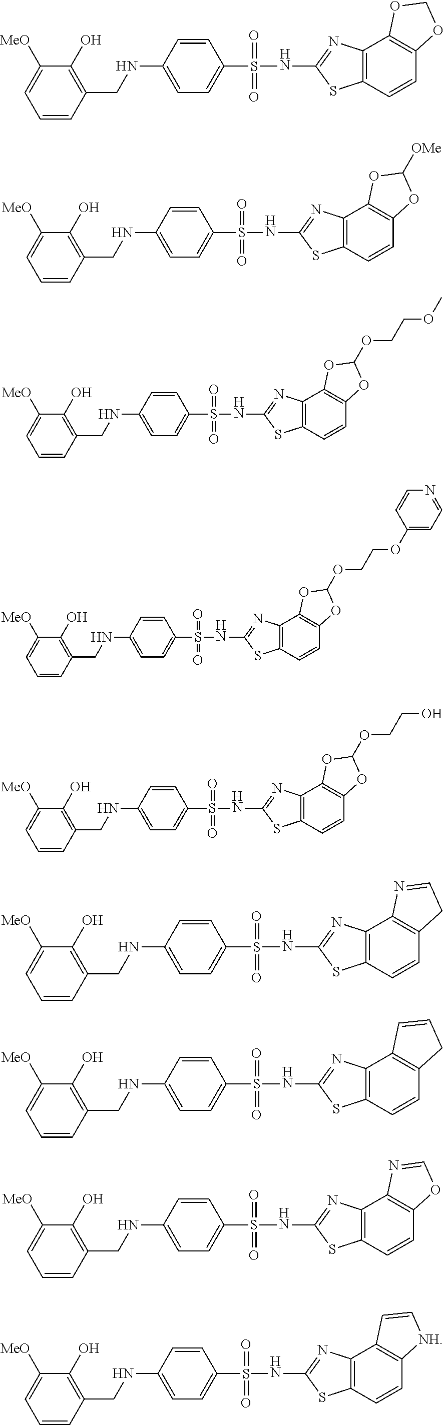

26. 4-((2-hydroxy-3-methoxybenzyl)amino)-N-(naphtho[1,2-d]thiazol-2-yl)benzenesulfonamide:

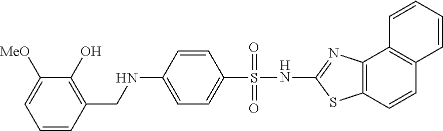 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.
27. A composition comprising:
   a compound of formula I:

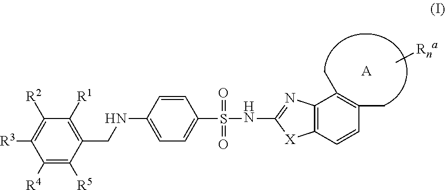

wherein R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof, and
a pharmaceutically acceptable excipient.
28. The composition according to 27, wherein each of R³, R⁴ and R⁵ are hydrogen.
29. The composition according to any one of 27-28, wherein R¹ is hydroxy.
30. The composition according to any one of 27-29, wherein R² is alkoxy.
31. The composition according to 30, wherein R² is methoxy.
32. The composition according to any one of 27-31, wherein X is S.
33. The composition according to any one of 27-32, wherein A is a six-membered ring.
34. The composition according to 33, wherein A is a six-membered heteroaryl ring.
35. The composition according to any one of 33-34, wherein the compound is of formula Ia:

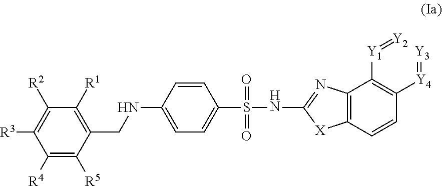

wherein Y₁, Y₂, Y₃ and Y₄ are each independently C or N.
36. The composition according to 35, wherein Y₁, Y₂, Y₃ and Y₄ are each C.
37. The composition according to 35, wherein:
   Y₃ is N; and
   Y₁, Y₂, and Y₄ are each C.
38. The composition according to 35, wherein:
   Y₁ and Y₄ are N; and
   Y₂ and Y₃ are C.
39. The composition according to any one of 33-38, wherein A is unsubstituted.
40. The composition according to any one of 33-38, wherein:
   n is 1 or 2; and
   each R^{a} is independently selected from:

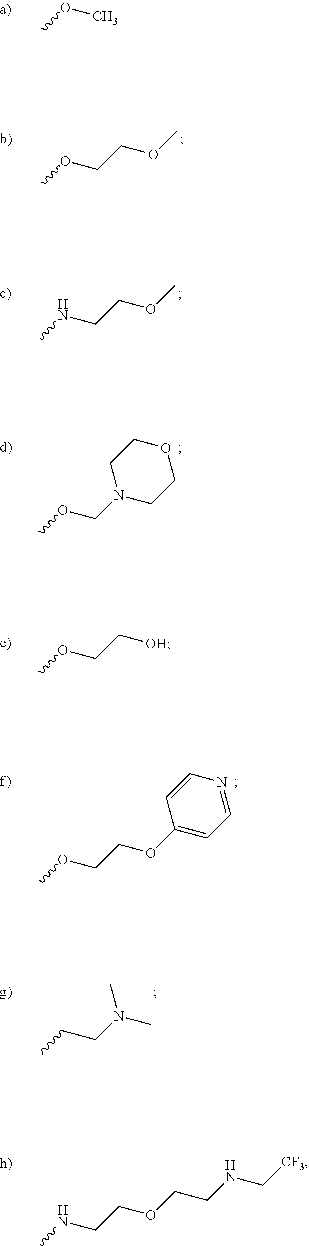

wherein [symbol] represents the A-R^{a} bond.
41. The composition according to any one of 33-40, wherein the compound is selected from:

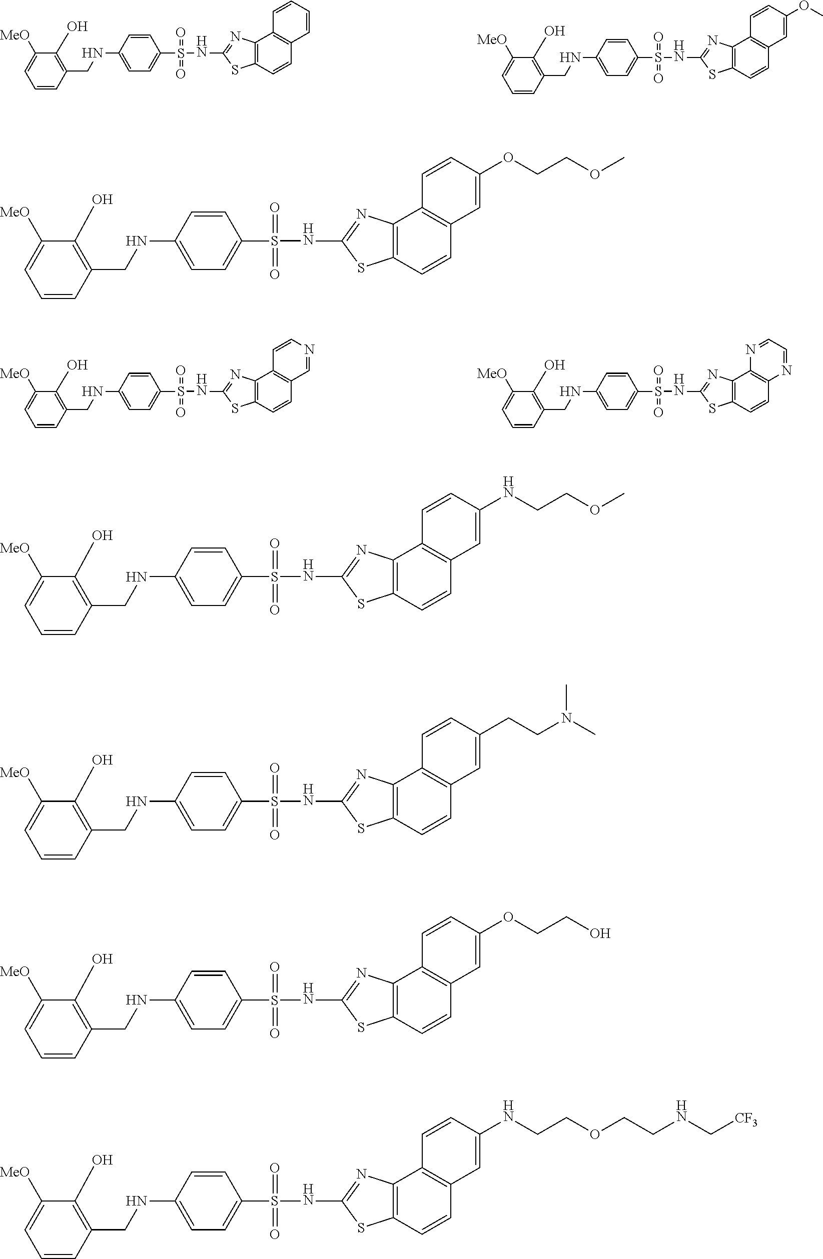

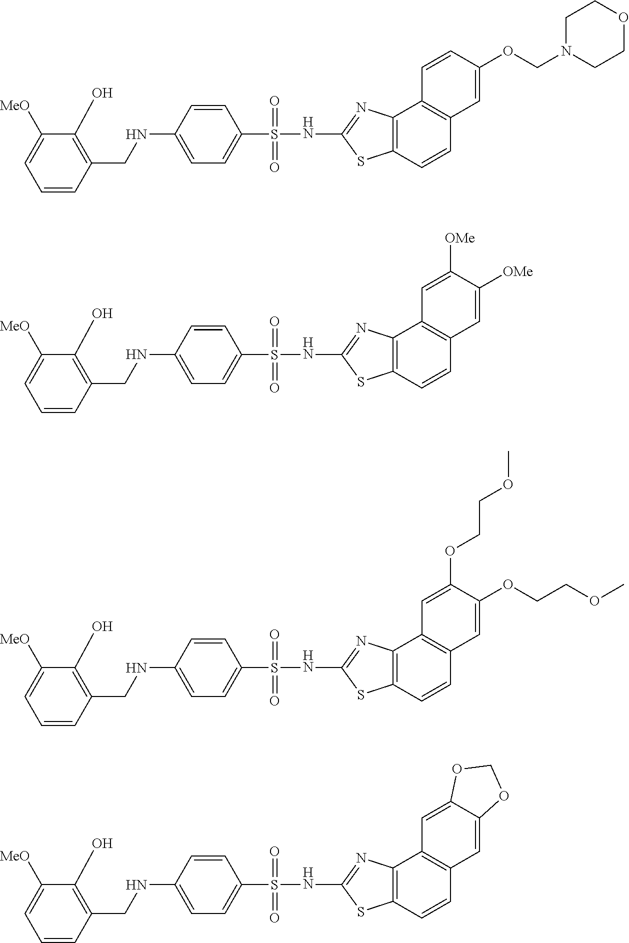 [SMILES: COc1cccc(CNc2ccc(S(=O)(=O)Nc3nc4c(ccc5cc(OCN6CCOCC6)ccc54)s3)cc2)c1O ; COc1cc2ccc3sc(NS(=O)(=O)c4ccc(NCc5cccc(OC)c5O)cc4)nc3c2cc1OC ; COCCOc1cc2ccc3sc(NS(=O)(=O)c4ccc(NCc5cccc(OC)c5O)cc4)nc3c2cc1OCCOC ; COc1cccc(CNc2ccc(S(=O)(=O)Nc3nc4c(ccc5cc6c(cc54)OCO6)s3)cc2)c1O]

42. The composition according to any one of 27-32, wherein A is a five-membered ring.
43. The composition according to 42, wherein A is a five-membered heterocyclic ring.
44. The composition according to 42, wherein A is a five-membered heteroaryl ring.
45. The composition according to any one of 42-44, wherein the compound is of formula Ib:

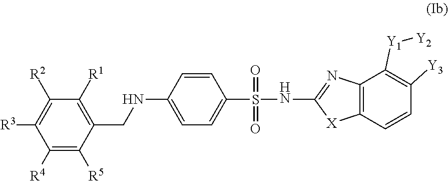

wherein Y₁, Y₂, Y₃ and Y₄ are each independently C, N or O.
46. The composition according to 45, wherein Y₁, Y₂ and Y₃ are each C.
47. The composition according to 45, wherein:
   Y₂ is C; and
   Y₁ and Y₃ are O.
48. The composition according to 45, wherein:
   Y₁ is N; and
   Y₂ and Y₃ are C.
49. The composition according to any one of 42-48, wherein A is unsubstituted.
50. The composition according to any one of 42-48, wherein:
   n is 1 or 2; and
   each R^{a} is independently selected from:

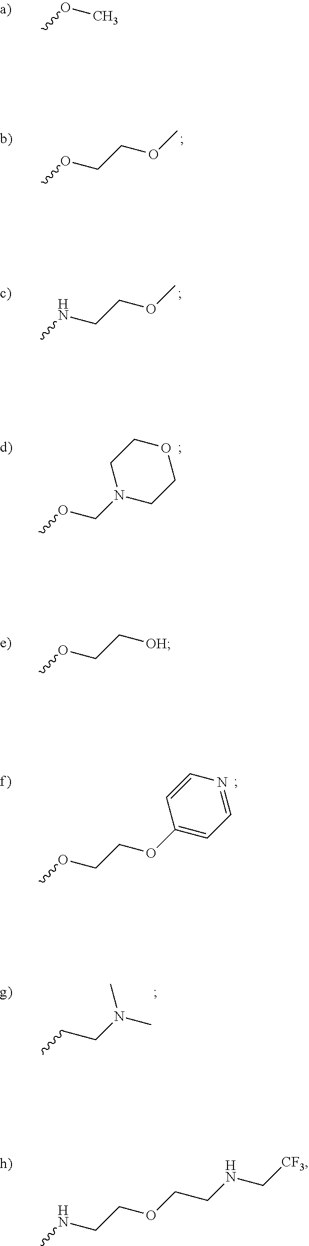

wherein [symbol] represents the A-R^{a} bond.
51. The composition according to any one of 42-50, wherein the compound is selected from:

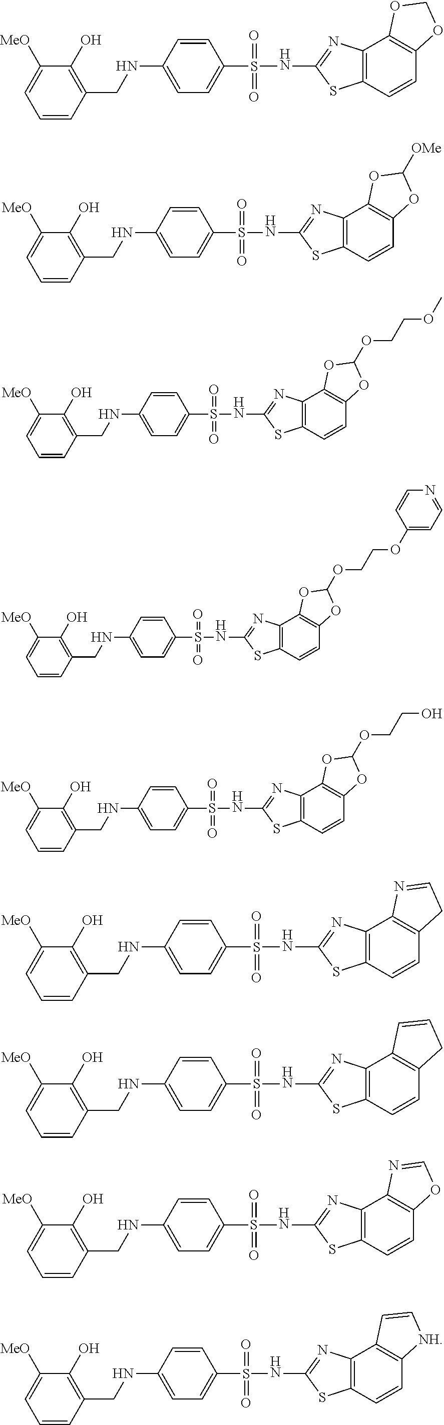

52. The composition according to 27, wherein the compound is 4-((2-hydroxy-3-methoxybenzyl)amino)-N-(naphtho[1,2-d]thiazol-2-yl)benzenesulfonamide:

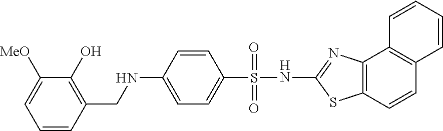 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.
53. A method for inhibiting human platelet 12-(S)-lipoxygenase, the method comprising contacting a cell with a compound of formula I:

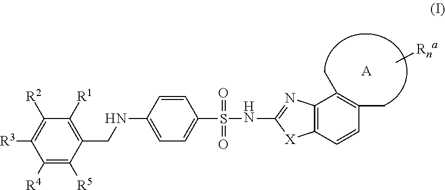

wherein R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof; and
a pharmaceutically acceptable excipient.
54. The method according to 53, wherein each of R³, R⁴ and R⁵ are hydrogen.
55. The method according to any one of 53-54, wherein R¹ is hydroxy.
56. The method according to any one of 53-55, wherein R² is alkoxy.
57. The method according to 56, wherein R² is methoxy.
58. The method according to any one of 53-57, wherein X is S.
59. The method according to any one of 53-58, wherein A is a six-membered ring.
60. The method according to 59, wherein A is a six-membered heteroaryl ring.
61. The method according to any one of 59-60, wherein the compound is of formula Ia:

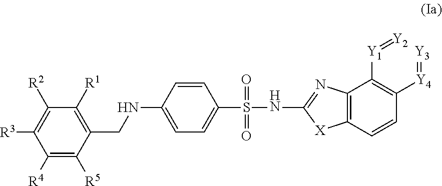

wherein Y₁, Y₂, Y₃ and Y₄ are each independently C or N.
62. The method according to 61, wherein Y₁, Y₂, Y₃ and Y₄ are each C.
63. The method according to 61, wherein:
   Y₃ is N; and
   Y₁, Y₂, and Y₄ are each C.
64. The method according to 61, wherein:
   Y₁ and Y₄ are N; and
   Y₂ and Y₃ are C.
65. The method according to any one of 59-64, wherein A is unsubstituted.
66. The method according to any one of 59-64, wherein:
   n is 1 or 2; and
   each R^{a} is independently selected from:

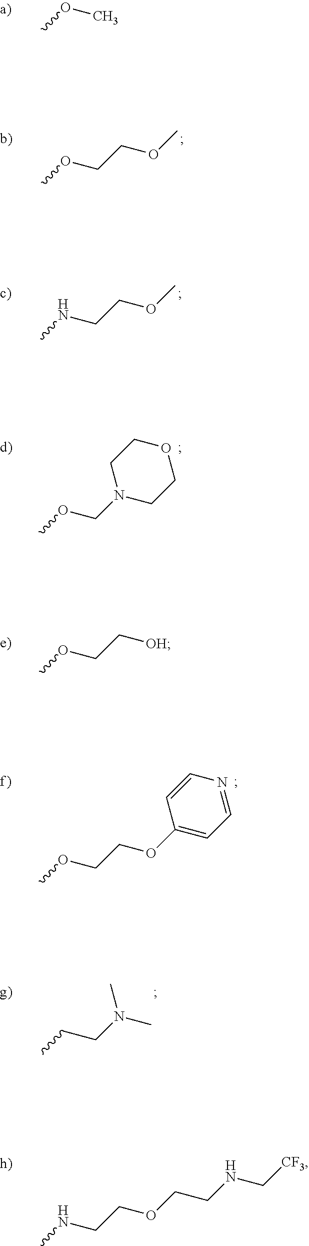

wherein [symbol] represents the A-R^{a} bond.
67. The method according to any one of 59-66, wherein the compound is selected from:

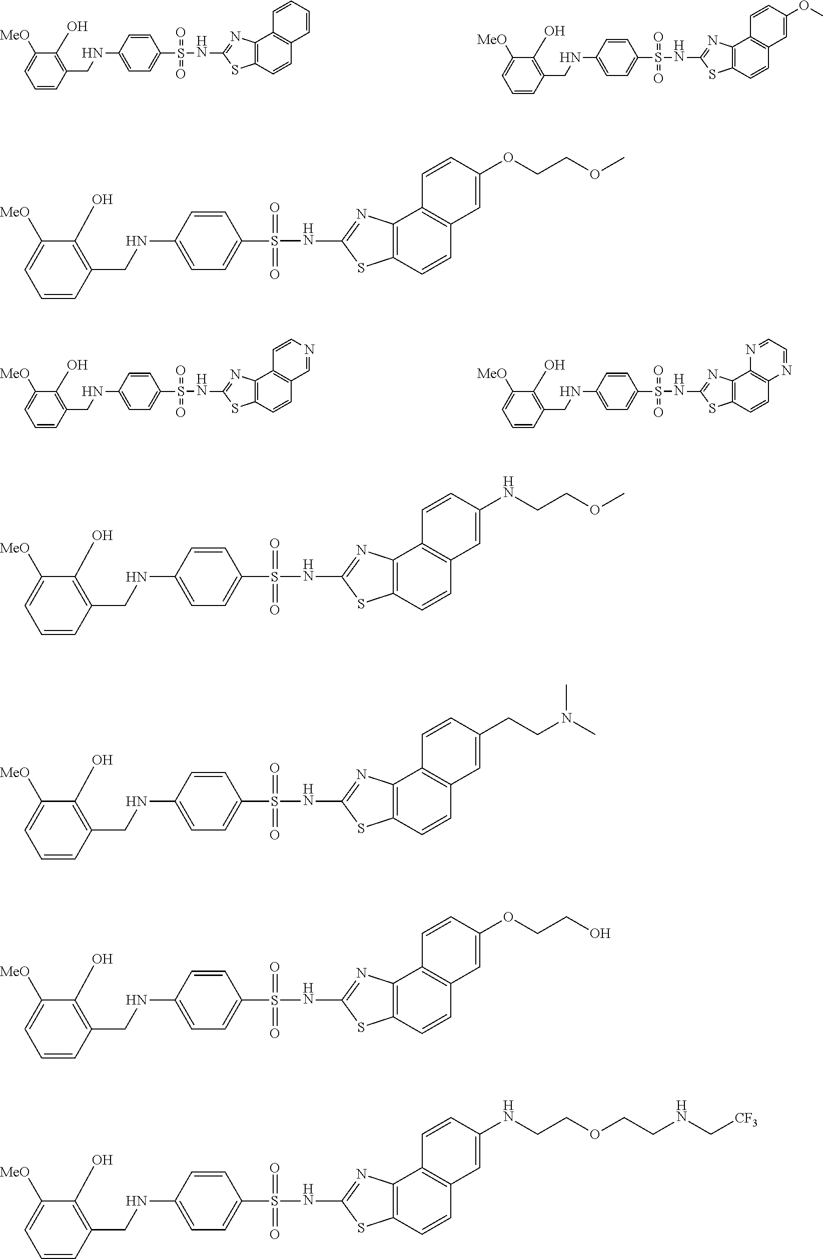

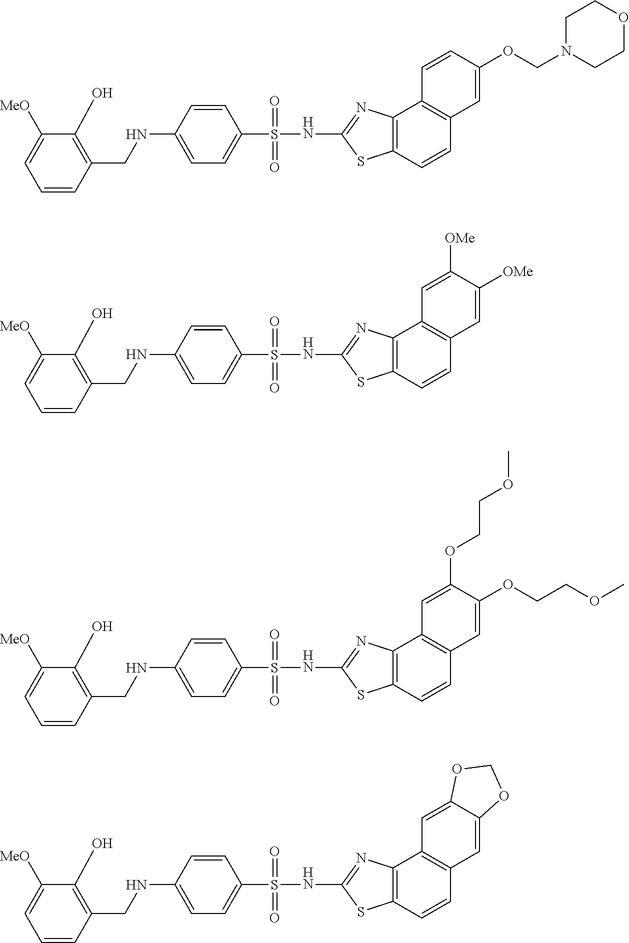 [SMILES: COc1cccc(CNc2ccc(S(=O)(=O)Nc3nc4c(ccc5cc(OCN6CCOCC6)ccc54)s3)cc2)c1O ; COc1cc2ccc3sc(NS(=O)(=O)c4ccc(NCc5cccc(OC)c5O)cc4)nc3c2cc1OC ; COCCOc1cc2ccc3sc(NS(=O)(=O)c4ccc(NCc5cccc(OC)c5O)cc4)nc3c2cc1OCCOC ; COc1cccc(CNc2ccc(S(=O)(=O)Nc3nc4c(ccc5cc6c(cc54)OCO6)s3)cc2)c1O]

68. The method according to any one of 53-58, wherein A is a five-membered ring.
69. The method according to 68, wherein A is a five-membered heterocyclic ring.
70. The method according to 68, wherein A is a five-membered heteroaryl ring.
71. The method according to any one of 68-70, wherein the compound is of formula Ib:

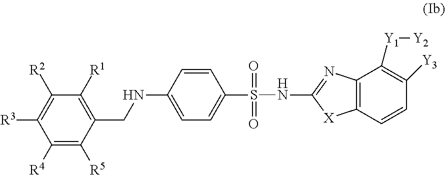

wherein Y₁, Y₂, Y₃ and Y₄ are each independently C, N or O.
72. The method according to 71, wherein Y₁, Y₂ and Y₃ are each C.
73. The method according to 71, wherein:
   Y₂ is C; and
   Y₁ and Y₃ are O.
74. The method according to 71, wherein:
   Y₁ is N; and
   Y₂ and Y₃ are C.
75. The method according to any one of 68-74, wherein A is unsubstituted.
76. The method according to any one of 68-74, wherein:
   n is 1 or 2; and
   each R^{a} is independently selected from:

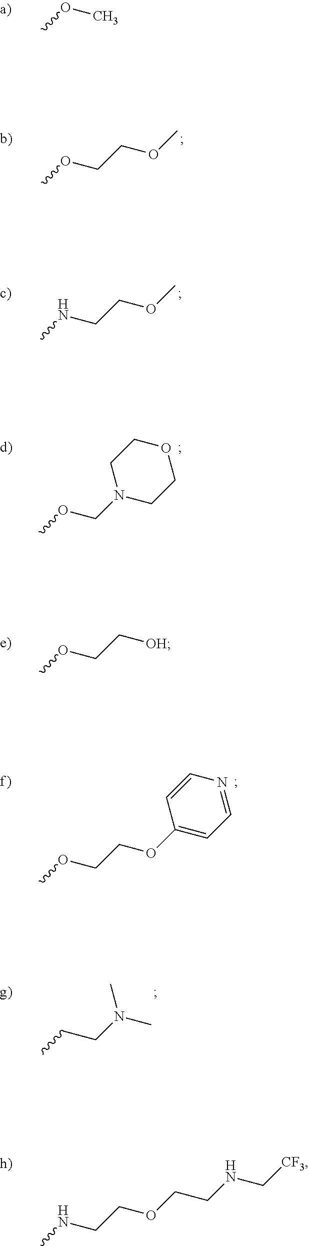

wherein [symbol] represents the A-R^{a} bond.
77. The method according to any one of 68-76, wherein the compound is selected from:

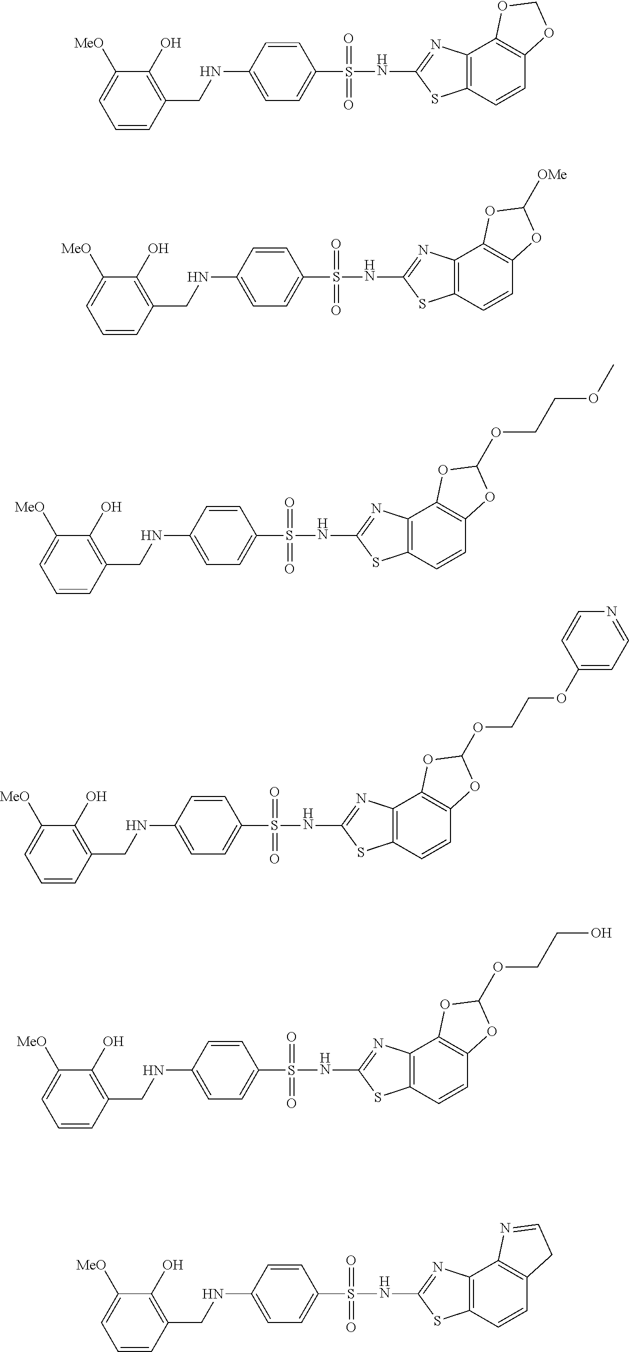 [SMILES: COc1cccc(CNc2ccc(S(=O)(=O)Nc3nc4c5c(ccc4s3)OCO5)cc2)c1O ; COc1cccc(CNc2ccc(S(=O)(=O)Nc3nc4c5c(ccc4s3)OC(OC)O5)cc2)c1O ; COCCOC1Oc2ccc3sc(NS(=O)(=O)c4ccc(NCc5cccc(OC)c5O)cc4)nc3c2O1 ; COc1cccc(CNc2ccc(S(=O)(=O)Nc3nc4c5c(ccc4s3)OC(OCCOc3ccncc3)O5)cc2)c1O ; COc1cccc(CNc2ccc(S(=O)(=O)Nc3nc4c5c(ccc4s3)OC(OCCO)O5)cc2)c1O ; COc1cccc(CNc2ccc(S(=O)(=O)Nc3nc4c5c(ccc4s3)CC=N5)cc2)c1O]

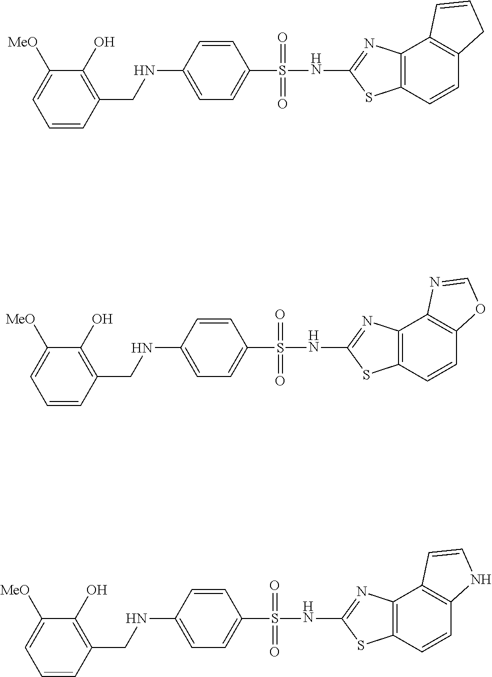 [SMILES: COc1cccc(CNc2ccc(S(=O)(=O)Nc3nc4c5c(ccc4s3)CC=C5)cc2)c1O ; COc1cccc(CNc2ccc(S(=O)(=O)Nc3nc4c(ccc5ocnc54)s3)cc2)c1O ; COc1cccc(CNc2ccc(S(=O)(=O)Nc3nc4c(ccc5[nH]ccc54)s3)cc2)c1O]

78. The method according to 53, wherein the compound is 4-((2-hydroxy-3-methoxybenzyl)amino)-N-(naphtho[1,2-d]thiazol-2-yl)benzenesulfonamide:

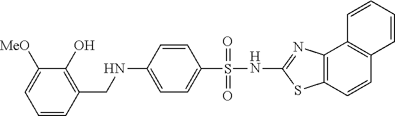 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.
79. A method comprising administering to a subject in need thereof a therapeutically effective amount of a compound of formula I:

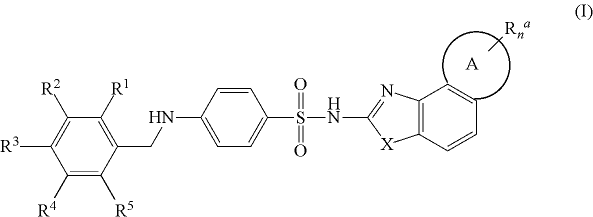

wherein R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof; and
a pharmaceutically acceptable excipient.
80. The method according to 79, wherein each of R³, R⁴ and R⁵ are hydrogen.
81. The method according to any one of 79-80, wherein R¹ is hydroxy.
82. The method according to any one of 79-81, wherein R² is alkoxy.
83. The method according to 82, wherein R² is methoxy.
84. The method according to any one of 79-83, wherein X is S.
85. The method according to any one of 79-84, wherein A is a six-membered ring.
86. The method according to 85, wherein A is a six-membered heteroaryl ring.
87. The method according to any one of 85-86, wherein the compound is of formula Ia:

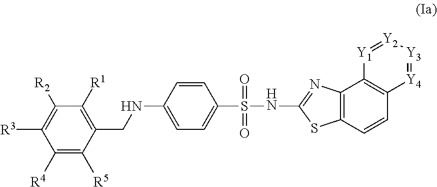

wherein Y₁, Y₂, Y₃ and Y₄ are each independently C or N.
88. The method according to 87, wherein Y₁, Y₂, Y₃ and Y₄ are each C.
89. The method according to 87, wherein:
   Y₃ is N; and
   Y₁, Y₂, and Y₄ are each C.
90. The method according to 87, wherein:
   Y₁ and Y₄ are N; and
   Y₂ and Y₃ are C.
91. The method according to any one of 85-90, wherein A is unsubstituted.
92. The method according to any one of 85-90, wherein:
   n is 1 or 2; and
   each R^{a} is independently selected from:

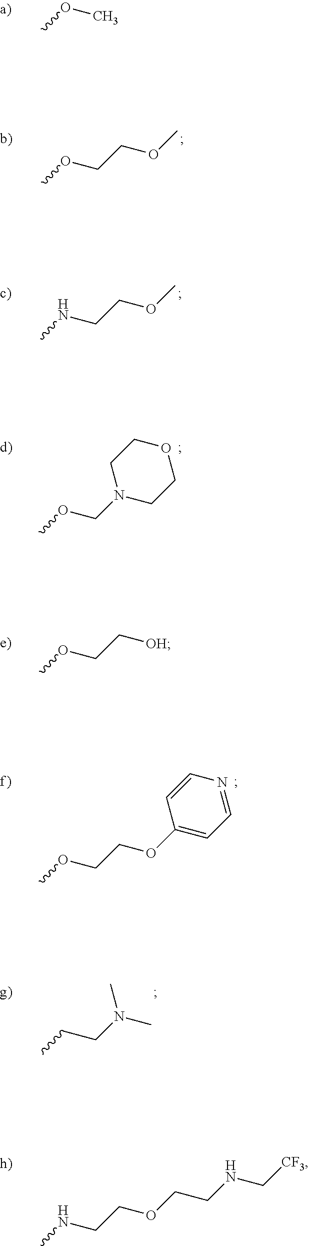

wherein [symbol] represents the A-R^{a} bond.
93. The method according to any one of 85-92, wherein the compound is selected from:

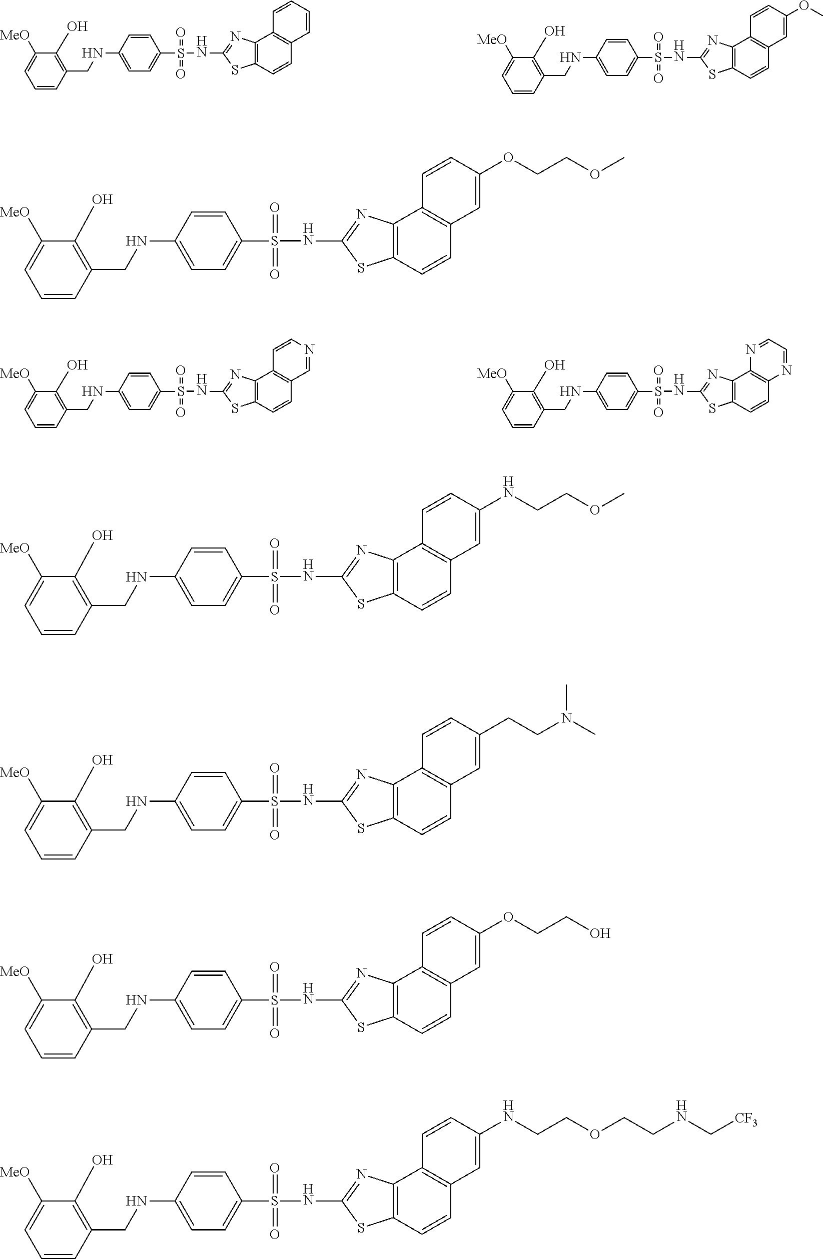

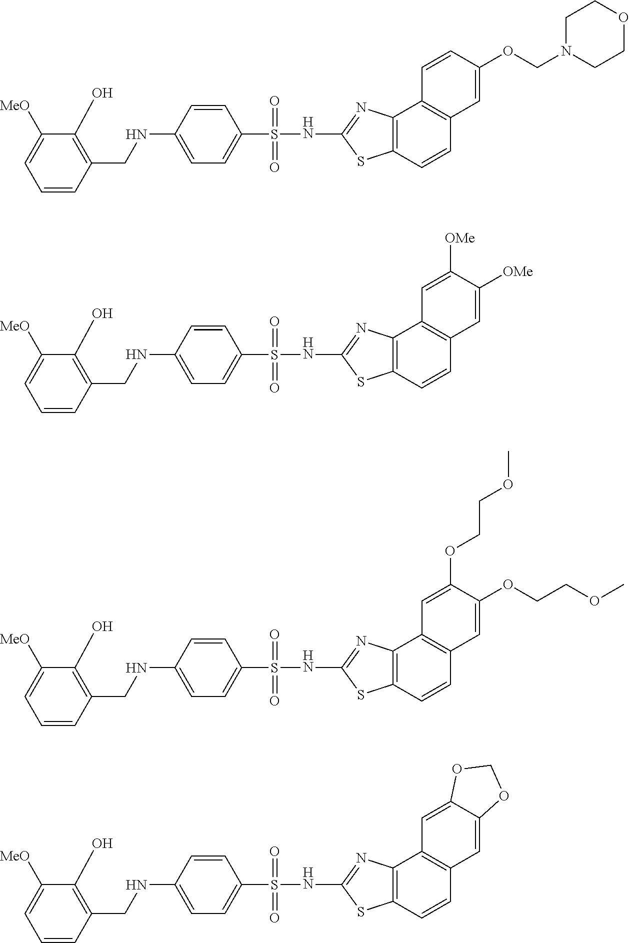 [SMILES: COc1cccc(CNc2ccc(S(=O)(=O)Nc3nc4c(ccc5cc(OCN6CCOCC6)ccc54)s3)cc2)c1O ; COc1cc2ccc3sc(NS(=O)(=O)c4ccc(NCc5cccc(OC)c5O)cc4)nc3c2cc1OC ; COCCOc1cc2ccc3sc(NS(=O)(=O)c4ccc(NCc5cccc(OC)c5O)cc4)nc3c2cc1OCCOC ; COc1cccc(CNc2ccc(S(=O)(=O)Nc3nc4c(ccc5cc6c(cc54)OCO6)s3)cc2)c1O]

94. The method according to any one of 79-84, wherein A is a five-membered ring.
95. The method according to 94, wherein A is a five-membered heterocyclic ring.
96. The method according to 94, wherein A is a five-membered heteroaryl ring.
97. The method according to any one of 94-96, wherein the compound is of formula Ib:

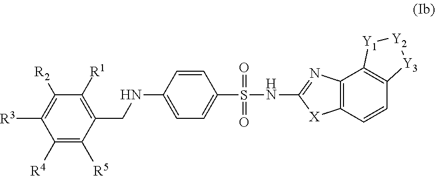

wherein Y₁, Y₂, Y₃ and Y₄ are each independently C, N or O.
98. The method according to 97, wherein Y₁, Y₂ and Y₃ are each C.
99. The method according to 97, wherein:
   Y₂ is C; and
   Y₁ and Y₃ are O.
100. The method according to 97, wherein:
   Y₁ is N; and
   Y₂ and Y₃ are C.
101. The method according to any one of 94-100, wherein A is unsubstituted.
102. The method according to any one of 94-100, wherein:
   n is 1 or 2; and
   each R^{a} is independently selected from:

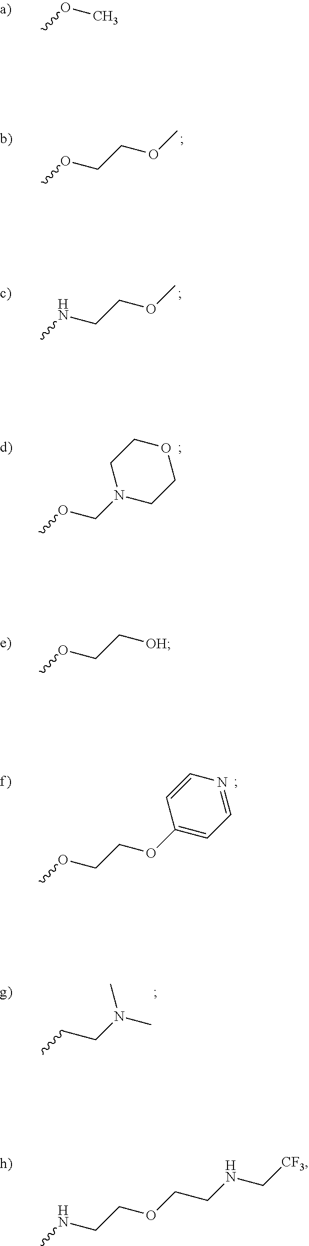

wherein [symbol] represents the A-R^{a} bond.
103. The method according to any one of 94-102, wherein the compound is selected from:

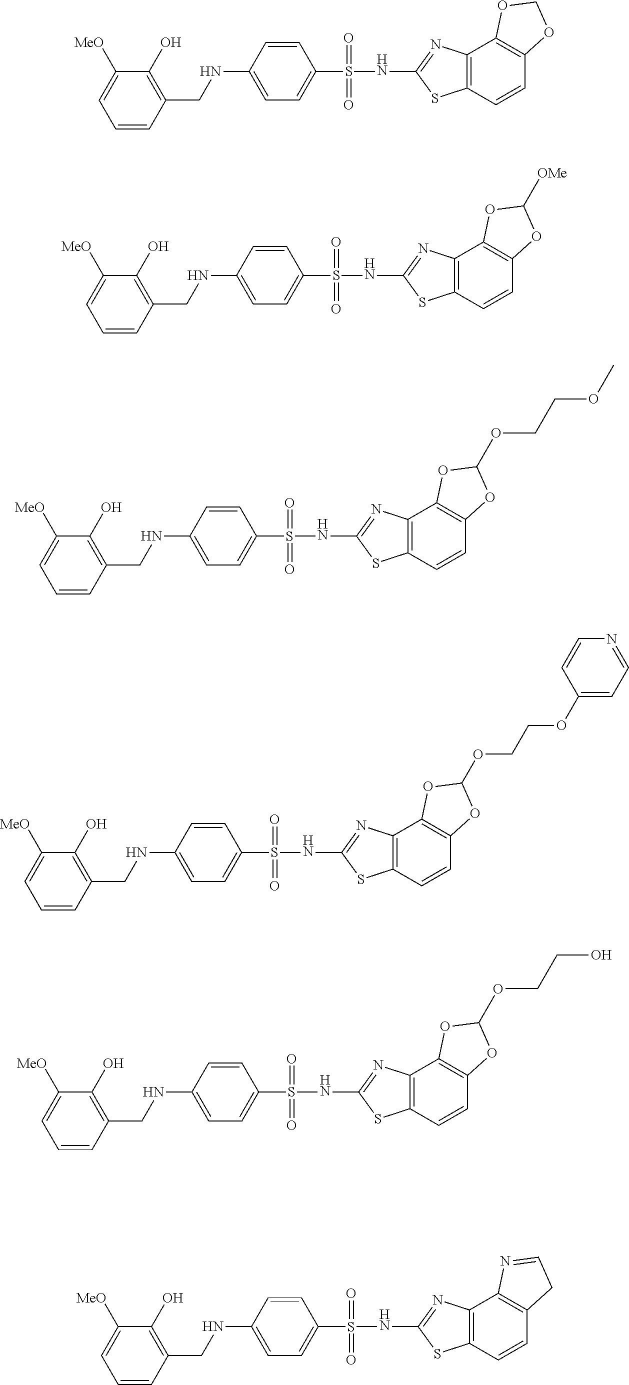 [SMILES: COc1cccc(CNc2ccc(S(=O)(=O)Nc3nc4c5c(ccc4s3)OCO5)cc2)c1O ; COc1cccc(CNc2ccc(S(=O)(=O)Nc3nc4c5c(ccc4s3)OC(OC)O5)cc2)c1O ; COCCOC1Oc2ccc3sc(NS(=O)(=O)c4ccc(NCc5cccc(OC)c5O)cc4)nc3c2O1 ; COc1cccc(CNc2ccc(S(=O)(=O)Nc3nc4c5c(ccc4s3)OC(OCCOc3ccncc3)O5)cc2)c1O ; COc1cccc(CNc2ccc(S(=O)(=O)Nc3nc4c5c(ccc4s3)OC(OCCO)O5)cc2)c1O ; COc1cccc(CNc2ccc(S(=O)(=O)Nc3nc4c5c(ccc4s3)CC=N5)cc2)c1O]

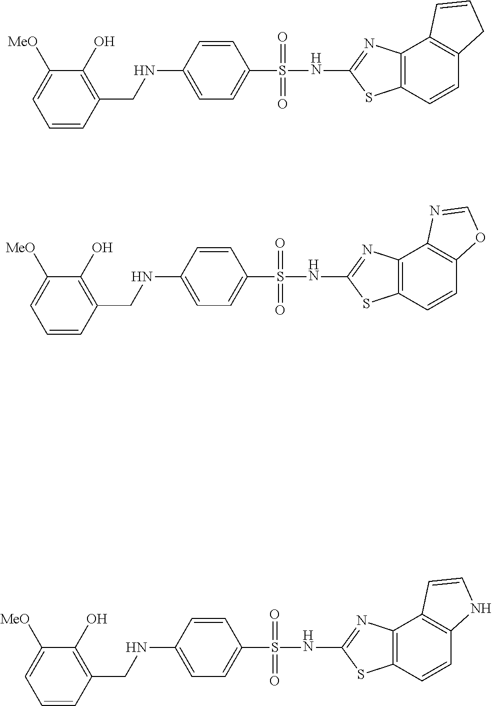 [SMILES: COc1cccc(CNc2ccc(S(=O)(=O)Nc3nc4c5c(ccc4s3)CC=C5)cc2)c1O ; COc1cccc(CNc2ccc(S(=O)(=O)Nc3nc4c(ccc5ocnc54)s3)cc2)c1O ; COc1cccc(CNc2ccc(S(=O)(=O)Nc3nc4c(ccc5[nH]ccc54)s3)cc2)c1O]

104. The method according to 79, wherein the compound is 4-((2-hydroxy-3-methoxybenzyl)amino)-N-(naphtho[1,2-d]thiazol-2-yl)benzenesulfonamide:

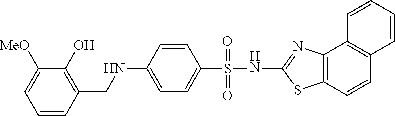 [SMILES: COc1cccc(CNc2ccc(S(=O)(=O)Nc3nc4c(ccc5ccccc54)s3)cc2)c1O]

or a pharmaceutically acceptable salt, solvate or hydrate thereof.
105. The method according to any one of 79-104, wherein the subject is diagnosed with a cardiovascular disease.
106. The method according to any one of 79-105, wherein the subject is diagnosed with diabetes.
107. The method according to 106, wherein the subject is diagnosed with Type I diabetes.
108. The method according to 106, wherein the subject is diagnosed with Type II diabetes.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the compounds of the present disclosure, it may be necessary and/or desirable to protect sensitive or reactive groups on any of the molecules concerned. This can be achieved by means of conventional protecting groups as described in standard works, such as T. W. Greene and P. G. M. Wuts, “Protective Groups in Organic Synthesis”, Fourth edition, Wiley, New York 2006. The protecting groups can be removed at a convenient subsequent stage using methods known from the art.

The compounds described herein can contain one or more chiral centers and/or double bonds and therefore, can exist as stereoisomers, such as double-bond isomers (i.e., geometric isomers), enantiomers or diastereomers. Accordingly, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

The nomenclature used herein to name the subject compounds is illustrated in the Examples herein. When possible, this nomenclature has generally been derived using the commercially-available AutoNom software (MDL, San Leandro, Calif).

### Materials and Methods

### Chemicals

Fatty acids used in this study were purchased from Nu Chek Prep, Inc. (MN, USA). All other solvents and chemicals were reagent grade or better and were used as purchased without further purification.

### Molecular Modeling

A homology model of the human platelet 12-LOX catalytic domain (Uniprot accession P18054) was built based on the porcine leukocyte 12(S)-LOX structure (PDB ID: 3RDE, sequence identity 65%), using the software Prime (version 6.0, Schrodinger Inc.). During homology modeling, the metal ion (Fe³⁺), the iron coordinated hydroxide ion and the co-crystallized ligand, 3-{4-[(tridec-2-yn-1-yloxy) methyl] phenyl} propanoic acid, from the porcine 12(S)-LOX structure were retained. In the published structure, a water is shown coordinating the metal ion; for modeling purposes, iron was assigned to the ferric (Fe³⁺) oxidation state, with a coordinating hydroxide to mimic the catalytically-competent state and complete the octahedral symmetry. The human 12-LOX model was energy minimized using Protein Preparation Wizard (Schrodinger Inc). During this step, hydrogen atoms were added and optimized to make better hydrogen bonding interactions, and all heavy atoms were energy minimized with a restraint such that they did not move beyond 0.3 Å from their starting positions. The structure of MHL355 was prepared using Edit/Build panel of Maestro software (version 12.4, Schrodinger Inc.) and energy minimized using LigPrep software (Schrodinger Software Suite 2020-2).

Initially, ML355 was docked to the wild-type (wt) protein using the docking software Glide (version 8.7, Schrodinger Inc.), consisting of grid preparation and virtual screening. The coordinates of the co-crystallized ligand from the porcine 12-LOX homolog were used to define the grid center. Following grid preparation, ML355 was docked to the h12-LOX active site using standard-precision (SP) scoring function. Only the ligand was treated flexibly during docking.

Starting from the wt protein structural model, each mutant protein was made using the Mutate Residue option in the Build panel of the Maestro software. After making the virtual mutation, sidechain conformation of the mutated residue was predicted using the software Prime (version 6.0, Schrodinger Inc.). During the sidechain prediction step, except for the metal and the hydroxide ions, all the other modeled compounds were removed from the active site. The protein was held rigid and only the conformation of the mutated residue was optimized. The coordinates of ML355 docked to the wt protein were used to define the docking grid center for each mutant protein. After the grid preparation step, MHL355 was docked to the mutant proteins using Glide with the SP scoring function.

### Site-Directed Mutagenesis

Based on the ML355 binding mode predicted by the modeling, all residues within 5 Å of any atom of ML355 were selected for experimental mutagenesis. The selected residues were replaced with amino acids with either bulkier or smaller side chains in order to determine what specific interactions play a role in inhibitor binding. The following mutations were made: F352L, 1399L, 1399M, E356A, E356K, L361M, R402L, A403S, L407A, L407G, 1413A, F414L, A417I, V418M, A417I/V418M, Q547L, and L597M. The numbering refers to the UniProt accession number P18054 sequence, with the N-terminal methionine assigned as amino acid number one. The primers for all mutants were designed using the online QuikChange Primer Design tool (http://www.genomics.agilent.com/primerDesignProgram.jsp) from Agilent Technologies (CA, USA). The mutations were introduced by using the QuikChange® II XL site-directed mutagenesis kit from Agilent, following the instructions in the protocol provided. The mutations were confirmed by sequencing the 12-LOX insert in the pFastBac1 shuttle vector (Eurofins Genomics, KY, USA).

### Protein Expression and Purification

The wild type 12-LOX (wt-12-LOX) enzyme and its mutants were expressed as fusion proteins, with an N-terminal 6×His tag, and were affinity purified by nickel-iminodiacetic acid agarose using FPLC (Biorad). For inhibitor selectivity, human 5-LOX (h5-LOX, UniProt entry P09917), human reticulocyte 15-LOX-1 (h15-LOX-1, UniProt entry P16050) and human epithelial 15-LOX-2 (h15-LOX-2, UniProt entry 015296) were also isolated. The purity of all the proteins, except h5-LOX, was greater than 90% as determined by SDS-PAGE analysis. h5-LOX was an ammonium sulfate fraction due to the extensive loss of activity upon purification.

### 2.5. Determination of Iron Content Using ICP-MS

The iron content of wt12-LOX and the mutant enzymes that exhibited a significant change in their IC₅₀ values was determined by a Thermo Element XR inductively-coupled plasma mass spectrometer (ICP-MS), using cobalt (EDTA) as an internal standard. Iron concentrations were compared with standardized iron solutions and all kinetic data were normalized to the iron content. The Bradford assay, with bovine serum albumin (BSA) as the protein standard, was used to determine the protein concentration.

### 2.6. Steady-State Kinetics

The kinetic rates of wt12-LOX and mutant enzymes that exhibited more than 3.5-fold increase in the IC₅₀ values were determined by monitoring the formation of the conjugated diene product, 12(S)-HpETE (ε=25,000 M⁻¹cm⁻¹) at 234 nm, with a Perkin-Elmer Lambda 40 UV/Vis spectrophotometer. The reactions were initiated by adding the appropriate amount of enzyme to a 2 mL reaction mixture containing 1-20 μM AA, 25 mM HEPES buffer (pH 8.0), 0.01% Triton X-100, at 23° C., with constant stirring using a magnetic bar. Kinetic data were obtained by recording initial enzymatic rates at each substrate concentration and then fitting them to the Michaelis-Menten equation using KaleidaGraph (Synergy) to determine the k_{cat} and k_{cat}/K_{M} values.

### 2.7. UV-Vis-Based IC₅₀ Assay

IC₅₀ values of ML355 against 12-LOX and its mutants were determined in a similar manner as the steady state kinetic values. The percent inhibition was determined by comparing the enzyme rates of the control (DMSO solvent) and the inhibitor sample by following the formation of the conjugated diene product at 234 nm (ε=25,000 M⁻¹cm⁻¹). The reactions were initiated by adding 200 nM 15-LOX-2, 30 nM 12-LOX, 40 nM 15-LOX-1, or approximately 100-300 nM (total protein) of 5-LOX ammonium sulfate fraction to a cuvette with 2 mL reaction buffer, constantly stirred using a magnetic stir bar at room temperature (22° C.). Reaction buffers used for various LOX isozymes were as follows: 25 mM HEPES (pH 7.3), 0.3 mM CaCl₂), 0.1 mM EDTA, 0.2 mM ATP, 0.01% Triton X-100, 10 μM AA for the crude, ammonium sulfate precipitated 5-LOX; and 25 mM HEPES (pH 7.5), 0.01% Triton X-100, 10 μM AA for 15-LOX-2, 15-LOX-1, and 12-LOX. The substrate concentration was quantitatively determined by allowing the enzymatic reaction to go to completion in the presence of soybean LOX-1. IC₅₀ values were obtained by determining the % inhibition, relative to solvent vehicle only, at various inhibitor concentrations. The data were then plotted against inhibitor concentration, followed by a hyperbolic saturation curve fit (assuming total enzyme concentration [E]<<IC₅₀). It should be noted that all of the potent inhibitors displayed greater than 80% maximal inhibition, unless otherwise stated in the tables. All inhibitors were stored at −20° C. in DMSO.

### Synthesis of 4-((2-hydroxy-3-methoxybenzyl)amino)-N-(naphtho[1,2-d]thiazol-2-yl)benzenesulfonamide (Compound LOX-12-001)

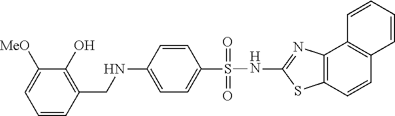 [SMILES: COc1cccc(CNc2ccc(S(=O)(=O)Nc3nc4c(ccc5ccccc54)s3)cc2)c1O]

### 4-((2-hydroxy-3-methoxybenzyl)amino)-N-(naphtho[1,2-d]thiazol-2-yl)benzenesulfonamide (Compound LOX-12-001)

All air or moisture sensitive reactions were performed under positive pressure of nitrogen with oven-dried glassware. Chemical reagents and anhydrous solvents were obtained from commercial sources and used as-is. The analog for assay has purity greater than 95% and ¹H and ¹³C NMR spectra were recorded on Bruker Avance III HD 500 MHz NMR spectrometer.

General Synthetic Procedures. The synthesis of the compounds described herein was achieved with the following steps (Scheme 1).

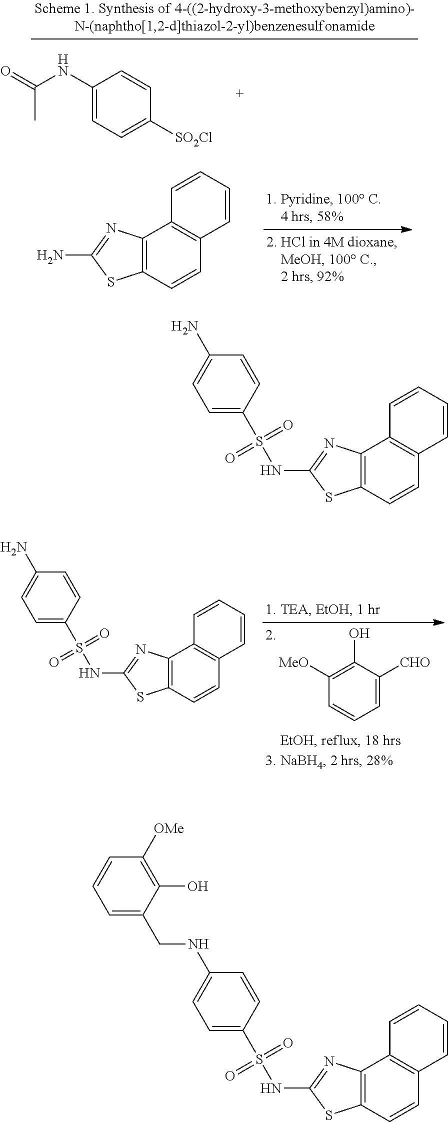

Step 1. To a stirred solution of naphtho[1,2-d]thiazol-2-amine (1 eq.) in pyridine (6 eq.) was added N-acetylsulfanilyl chloride (1.1 eq.) in three equal parts. The reaction mixture was heated for 4 hrs at 100° C. and allowed to cool to room temperature. The reaction mixture was then allowed to sit at room temperature for 2 hrs, and afterwards was poured into EtOAc and 1N HCl_{(aq)} in a separation funnel. A white solid was formed in the EtOAc layer, which was collected by filtration, washed with cold ethanol, and dried under reduced pressure overnight to give the desired acetamide product (yield: 58%).

Step 2. To a stirred solution of the acetamide product (1 eq.) in MeOH (2 mL) was added 4M HCl in dioxane (1.5 eq.). The reaction was heated for 2 hrs at 100° C. and then cooled to room temperature. The reaction mixture was then concentrated with a rotavap to get the white solid amine product (yield: 92%).

Step 3. The amine product (1 eq.) in anhydrous ethanol (1 mmol of amine product in 5 mL) was added to TEA (2 eq.) and stirred for 1 hr at room temperature. 2-hydroxy-3-methoxybenzaldehyde (1.2 eq.) was then added to the reaction mixture and refluxed overnight. The reaction mixture was then cooled to room temperature before sodium borohydride (3 eq.) was added and stirred for 2 hours. The reaction was quenched with MeOH and water until it stopped bubbling. The white solid was collected by filtration, washed with cold EtOH and then dried under vacuum. The crude solid was purified by RP-18 column chromatography (3 column volumes of 20% MeOH w/0.1% NH₄OH in water to wash out the impurity then elute out the product with 50% MeOH w/0.1% NH₄OH in water) to give the desired final product (yield: 28%).

### 4-((2-hydroxy-3-methoxybenzyl)amino)-N-(naphtho[1,2-d]thiazol-2-yl)benzenesulfonamide

¹H NMR (500 MHz, DMSO-d₆) δ 8.69 (s, 1H), 8.34 (d, J=5 Hz, 1H), 7.93 (s, 1H), 7.77 (s, 1H), 7.53 (d, J=10 Hz, 4H), 6.81 (d, J=10 Hz, 1H), 6.73 (d, J=10 Hz, 1H), 6.66 (t, J=5 Hz, 1H), 6.57 (d, J=5 Hz, 2H), 4.21 (d, J=10 Hz, 2H), 3.77 (s, 3H). ¹³C NMR (500 MHz, DMSO-d₆) δ 147.1, 143.5, 131.3, 127.6, 127.5, 127.3, 125.6, 119.8, 119.1, 118.4, 110.3, 110.1, 55.6, 40.6.

### Culture of Human Islets

Human islets from nondiabetic donors were received from the Integrated Islet Distribution Program or Prodo Laboratories (Aliso Viejo, CA) (Table 1). Characteristics of islets and donors are summarized in the below table. Islets were incubated in CMRL-1066 medium supplemented with 10% fetal bovine serum and 1% Pen-Strept overnight at 37° C. in 5% CO₂ for recovery from shipment. On the next day, islets were transferred to CMRL-1066 supplemented with 5% fetal bovine serum and 1% Pen-Strept. A portion of the islets were randomly selected and incubated with a mixture of human PICs, 0.57 mmol/L tumor necrosis factor α, 5.9 mmol/L interferon-γ, and 0.29 mmol/L IL-1β (all from BD Biosciences, San Jose, CA) in the presence or absence of 1 μmol/L compound LOX-12-001. Compound LOX-12-001 was added 30 minutes before the addition of pro-inflammatory cytokines (PIC).

TABLE 1
Characteristics of Human Islet Donors

| | | BMI | | Cause of Death, | | |
|---|---|---|---|---|---|---|
| | Age | (kg/ | | A1c Value | Study | |
| Sex | (y) | m²) | Race | if Reported | Done | Source |
| F | 60 | 44.4 | Wh | Head Trauma | Nondiabetic | IIDP |
| | | | | | donors | |
| F | 53 | 32.5 | Cau- | Anoxic event, | Nondiabetic | Prodo |
| | | | casian | HbA1c 5.2% | donors | |
| M | 34 | 28.1 | His- | Head trauma | Nondiabetic | IIDP |
| | | | panic | | donors | |

### Glucose Stimulated Insulin Secretion

Use of Krebs-Ringer buffer (KRB) for glucose-stimulated insulin secretion (GSIS) was previously published. For batch assays, islets treated with PIC in the presence or absence of compound LOX-12-001 (1 μM) for 24 hours were transferred to KRB without glucose and were incubated for 1 hour at 37° C. in 5% CO₂ in the absence of PIC and compound LOX-12-001. Thereafter, 50 islet equivalents (IEQs) per well were transferred to six-well plates filled with 1 mL of KRB containing 3 mmol/L or 18 mmol/L of glucose and were incubated for 1 hour at 37° C. in 5% CO₂. Each condition was performed in triplicate. After incubation, supernatant was collected for determination of insulin secretion by human insulin enzyme-linked immunosorbent assay (Mercodia, Winston-Salem, NC).

### Statistics

Data are presented as mean±standard error of the mean and were analyzed using Two-way Annova analysis of variance Sidak's multiple comparison test (Prism software, Irvine, CA) as indicated in each figure. P<0.05 was considered significant.

### Results and Discussion

Computational Modeling of ML355 Interactions with wt12-LOX and its Mutants.

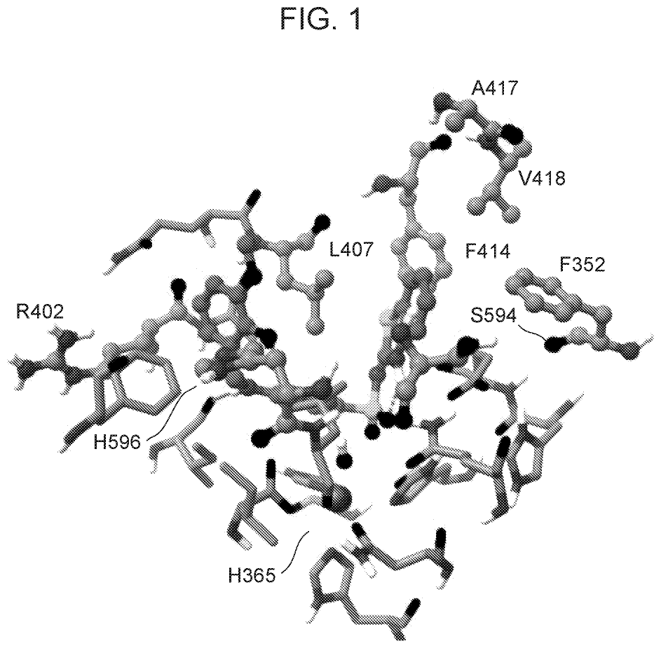

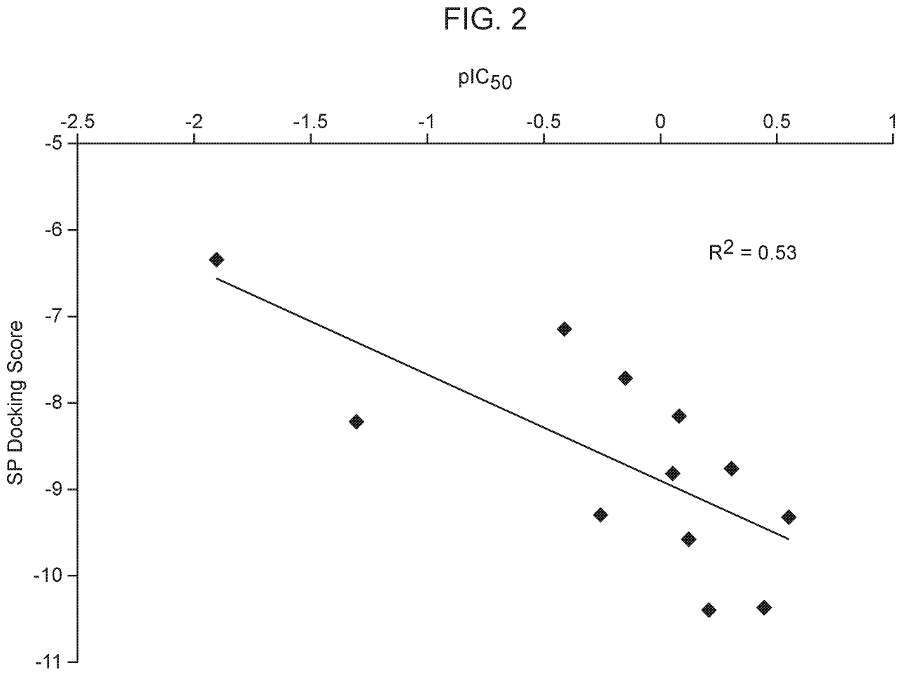

MHL355 is a potent/selective inhibitor of h12-LOX, however little is known regarding its specific molecular interactions in the active site of h12-LOX. In order to rectify this deficiency, a homology model of the h12-LOX catalytic domain was constructed and ML355 was docked into the catalytic site. Modelling predicted MHL355 to bind tightly in the active site with a standard-precision (SP) docking score of −10.37. The docking pose (FIG. 1) shows the following interactions between ML355 and h12-LOX. The amidine hydrogen forms a hydrogen bond with the backbone carbonyl oxygen of 1399, the bridging aromatic ring forms a pi-stacking interaction with the metal coordinated H365, the epsilon nitrogen of H596 is 3.6 Å from the oxygen atom of the hydroxyl group on the p-methoxy catechol moiety, ML355 wraps around L407 and the ML355 benzothiazole ring is buried in a hydrophobic pocket, whose base is defined by A417/V418 and whose sides are defined by the aromatic residues, F352 and F414. Based on this docking model, homology models were constructed of critical interaction mutations and docking calculations were performed for ML355 binding (Table 2). In parallel, the h12-LOX mutants were purified and their IC₅₀ values determined to assess the validity of the docking model. The docking scores show a positive correlation with the experimental IC₅₀ values (FIG. 2).

TABLE 2
ML355 IC₅₀ values and XP docking scores of the
wt12-LOX and the mutant enzymes.

| | | SP Docking |
|---|---|---|
| Enzyme | ML355 IC₅₀ (μM) | Score |
| wt12-LOX | 0.36 ± 0.02 | −10.4 |
| R402L | 0.28 ± 0.06 | −9.32 |
| H596L | 1.8 ± 0.4 | −9.29 |
| L407A | 1.4 ± 0.18 | −7.71 |
| L407G* | 2.6 ± 0.86 | −7.14 |
| A417I | 0.62 ± 0.08 | −10.4 |
| V418M | 0.76 ± 0.07 | −9.57 |
| A417I/V418M | 0.83 ± 0.2 | −8.14 |
| A417I/V418M/S594T | >20 | −8.23 |
| A417I/V418M/L407G | >80 | −6.34 |
| F414L | 0.49 ± 0.03 | −8.75 |
| F352L | 0.89 ± 0.08 | −8.81 |

*The high error for L407G is due to is low absolute activity.

### Role of L407 in ML355 Positioning

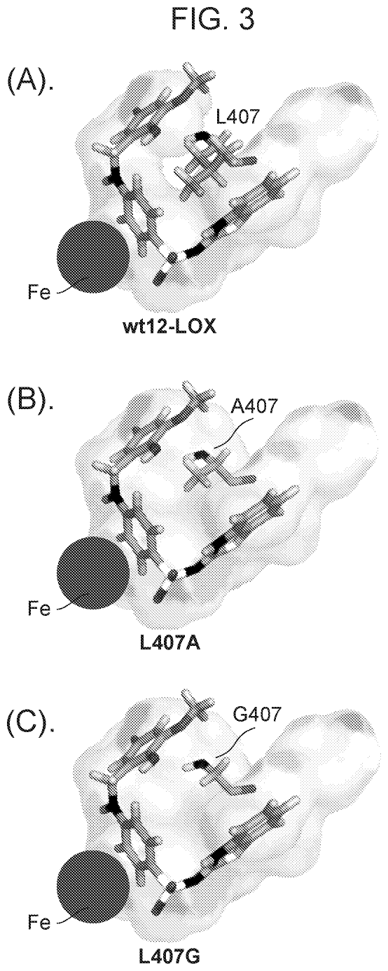

On the basis of the computational model of ML355 in the active site, the importance of the bulky hydrophobic residue, L407, was investigated. Our ML355 docking model indicated that ML355 wrapped around L407 and replacement of L407 with a smaller alanine residue caused a 3.9-fold decrease in inhibitory potency compared to wt12-LOX, 1.4±0.18 μM and 0.36±0.02 μM, respectively. This change in inhibitory potency decreased even more when the cavity was made larger with the L407G mutant enzyme, with a 7.1-fold decrease in IC₅₀ relative to wt12-LOX, 2.6±0.9 μM and 0.36±0.02 μM, respectively. The side chain of L407, along with 1399, A403, L597, form a narrow hydrophobic channel for the substrate. As shown in FIG. 1, the phenyl ring linker of MHL355 fills the hydrophobic channel in the wt-12-LOX binding pose. Mutation of L407 to a smaller hydrophobic residue, such as Ala or Gly, greatly widens the hydrophobic channel, reducing favorable hydrophobic contacts with ML355, which in turn would affect ML355 potency (FIG. 3).

### Role of A417, V418 and S594 in ML355 Binding

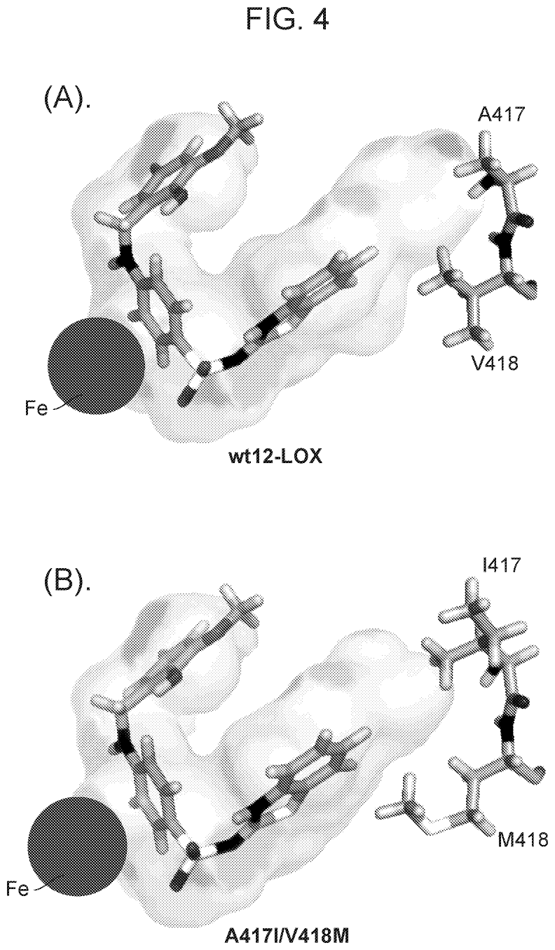

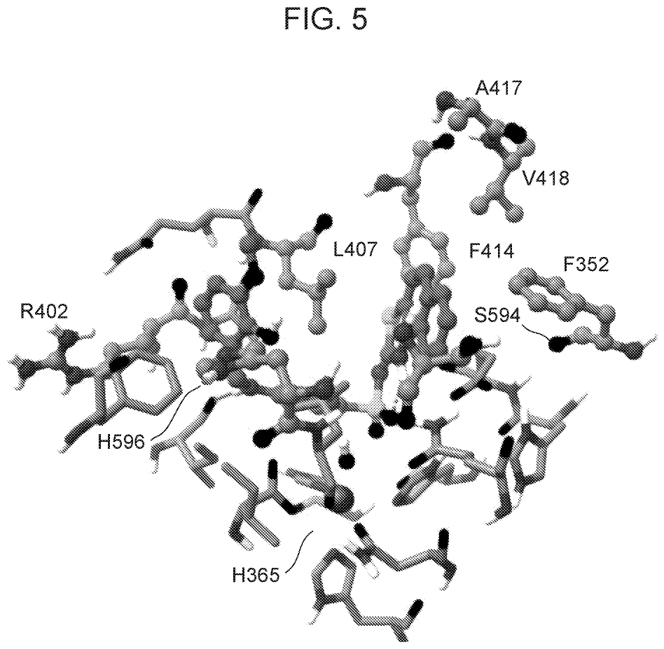

Residues A417 and V418 (FIG. 1), known as the Sloane determinants, form the bottom of the active site cavity and play a major role in the substrate positional specificity of 12-LOX. However, the docking model did not indicate a structural interaction between these two residues and the inhibitor (FIG. 4, panel A and panel B). The benzothiazole of ML355 was positioned 6.9 Å away from A417 and 4.2 Å away from V418. Therefore, the mutants, A417I, V418M, and A417I/V418M, were tested to determine if the decreased depth of the active site would affect ML355 potency. As predicted by our docking model, mutating these two residues to bulkier amino acids showed only a modest change of the IC₅₀ value compared to the wt-12LOX, with only a 2.3-fold decrease in potency in the double mutant (A417I/V418M) (FIG. 4, Table 2). This result indicated that bulkier substituents on the benzothiazole ring of ML355 could be tolerated and possibly increase the ML355 derivative potency by increased hydrophobic interactions in the active site. Therefore, 4-((2-hydroxy-3-methoxybenzyl)amino)-N-(naphtho[1,2-d]thiazol-2-yl)benzenesulfonamide (Compound LOX-12-001) was docked to the h12-LOX active site and found to have a favorable binding score of −11.40 (FIG. 5). The molecule was subsequently synthesized (Scheme 1) and its potency was observed to increase by 7.2-fold (IC₅₀=0.050±0.02 μM) (Table 3) against the wt12-LOX.

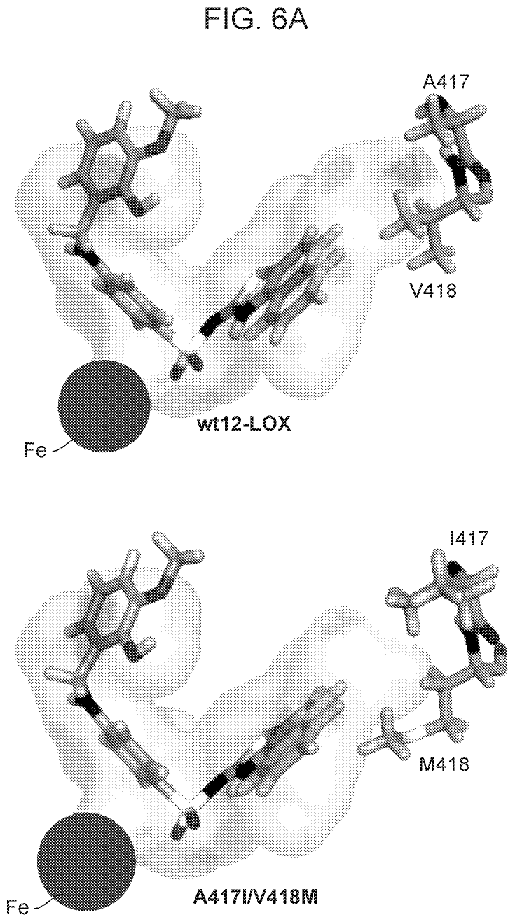

When compound LOX-12-001 was screened against the A417I/V418M mutant, the potency only decreased 2.4-fold, similar to that seen with ML355, indicating additional cavity area near the (naphthyl)thiazole moiety of compound LOX-12-001 (FIG. 6). AA catalysis was observed that to affect substrate binding, it was necessary to not only shorten the active site cavity (A417I/V418M) but to also make it narrower (S594T). The triple mutant, A417I/V418M/S594T, restricted the methyl tail of AA sufficiently to increase 15-HETE production significantly. Both ML355 and compound LOX-12-001 were tested against A417I/V418M/S594T and observed a dramatic decrease in potency for both inhibitors (greater than 20 μM IC₅₀ values for both ML355 and compound LOX-12-001 (Table 3)). These results indicate that by reducing both the active site depth and width, the inhibitor potency is reduced, supporting our inhibitor docking model.

As discussed above, L407 plays a role in ML355 binding by interacting with its phenyl ring linker and positioning the ends of ML355 properly in the “U” shaped cavity. It was therefore postulated that mutating L407 could have an additive effect with other mutants and thus the triple mutant, L407G/A417I/V418M, was designed to investigate the effect of both widening and reducing the length of the active site cavity on ML355 positioning. Amazingly, this triple mutant lowered the inhibitor potency dramatically for both ML355 and compound LOX-12-001 with IC₅₀ values of greater than 80 and 15 uM, respectively (Table 3), indicating that without the bulk of L407, a more shallow active site impedes inhibitor binding. Finally, it should be noted that the IC₅₀ value trend of compound LOX-12-001 against the mutants, A417I/418M, A417I/V418M/S594T and L407G/A417I/V418M (Table 3), are similar to the IC₅₀ value trend of ML355, suggesting a similar binding mode between compound LOX-12-001 and ML355.

TABLE 3
IC₅₀ values of (A) ML355 and (B) Compound LOX-12-001
with wt12-LOX and the mutants of Sloane determinants.

| | | Compound |
|---|---|---|
| | ML355 | LOX-12-001 |
| Enzyme | (μM) | (μM) |
| wt12-LOX | 0.36 ± 0.02 | 0.05 ± 0.02 |
| A417I/V418M | 0.83 ± 0.18 | 0.12 ± 0.04 |
| A417I/V418M/S594T | >20 (45% ^{a}) | >20 (44 % ^{a}) |
| L407G/A417I/V418M | >80 (19% ^{a}) | >150 (10 % ª) |

ª Percent inhibition at 20 uM inhibitor.

π-π Interactions of ML355 and Compound LOX-12-001 with F352 and F414 12-LOX

The ML355 docking model predicts that the benzothiazole ring of ML355 is buried in a hydrophobic pocket created by the aromatic side chains of F352 and F414, with closest distances to ML355 being 4.1 Å and 4.5 Å, respectively (FIG. 1). Since compound LOX-12-001 has a similar binding mode to ML355 in the 12-LOX active site, the aromatic residues participate in π-π interactions with the benzothiazole moieties of both ML355 and compound LOX-12-001. Therefore, the mutants, F352L and F414L, were generated, however for ML355, only a modest increase in IC₅₀ was observed, compared to wt12-LOX (Table 4). On the other hand, the IC₅₀ values of compound LOX-12-001 against these mutants increased more significantly, with a 3.6-fold increase for F414L and a 9.8-fold increase for F352L (Table 4). These results are intriguing because both mutations increase the active site cavity size, however they have a greater negative effect on the larger inhibitor, compound LOX-12-001. This suggests that there is a specific interaction with compound LOX-12-001 that is not present in ML355, which is disrupted upon the decreased size and loss of aromaticity in the active site mutations. Since compound LOX-12-001 is not only larger but has a more extensive aromatic structure than ML355, the data is consistent with compound LOX-12-001 having a stronger π-π interaction with F352 and F414 than ML355, which is lost upon mutation.

TABLE 4
IC₅₀ values of wt12-LOX, F414L and F352L with ML355 and
compound LOX-12-001 to investigate π-π interactions.

| | ML355 | Compound LOX- |
|---|---|---|
| Enzyme | (μM) | 12-001 (μM) |
| wt12-LOX | 0.36 ± 0.02 | 0.05 ± 0.02 |
| F414L | 0.49 ± 0.03 | 0.18 ± 0.01 |
| F352L | 0.89 ± 0.08 | 0.49 ± 0.05 |

H596 Binding Interaction with the p-Methoxy Catechol Moiety of ML355

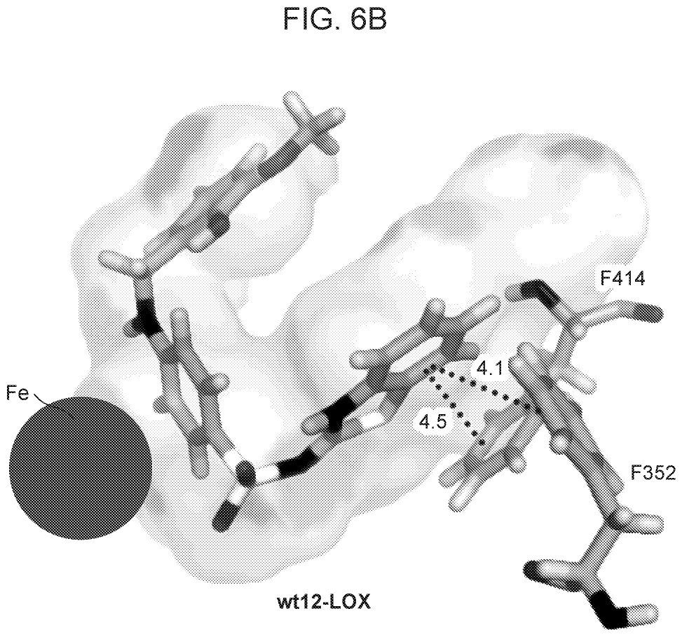

The role of R402 was previously determined unimportant with respect to substrate binding for h12-LOX. In this work, R402 was also determined to not affect ML355 binding (Table 2). However, computational prediction and subsequent experimental study showed that H596 did play a role in anchoring the carboxylate of AA during catalysis. Considering this result and close proximity (3.6 Å) of the H596 epsilon nitrogen (NE) to the oxygen atom of the hydroxyl group of p-methoxy catechol moiety of ML355 (FIG. 7A), H596L was generated and displayed a 5-fold decrease in potency relative to wt12-LOX, 1.8±0.4 μM and 0.36±0.02 μM, respectively (Table 2). These results suggest that H596 in h12-LOX may contribute to properly positioning ML355 in the active site, possibly through the interaction with the p-methoxy catechol moiety of ML355, as observed in the docking model. This can be further supported by the observed lowest energy docking pose of ML355 against H596L mutant, in which the p-methoxy catechol moiety is 1800 flipped from the pose observed for the wild-type (FIGS. 7A and 7B). Finally, it should be noted that an additional 7 mutations of active site residues were generated, however, they did not affect the potency of ML355 and thus were not investigated further (Table 5).

TABLE 5
The IC₅₀ values of additional active site mutations.

| Enzyme | ML355 IC50 (μM) |
|---|---|
| wt12-LOX | 0.36 ± 0.02 |
| L361M | 0.73 ± 0.06 |
| I399L | 0.83 ± 0.07 |
| E356A | 0.76 ± 0.08 |
| A403S | 0.61 ± 0.04 |
| I413A | 0.66 ± 0.06 |
| Q547L | 0.51 ± 0.02 |
| L597M | 1.1 ± 0.1 |

Islet Effect: Compound LOX-12-001 Improves GSIS and Increases Insulin Secretion of Human Islets Treated with PIC

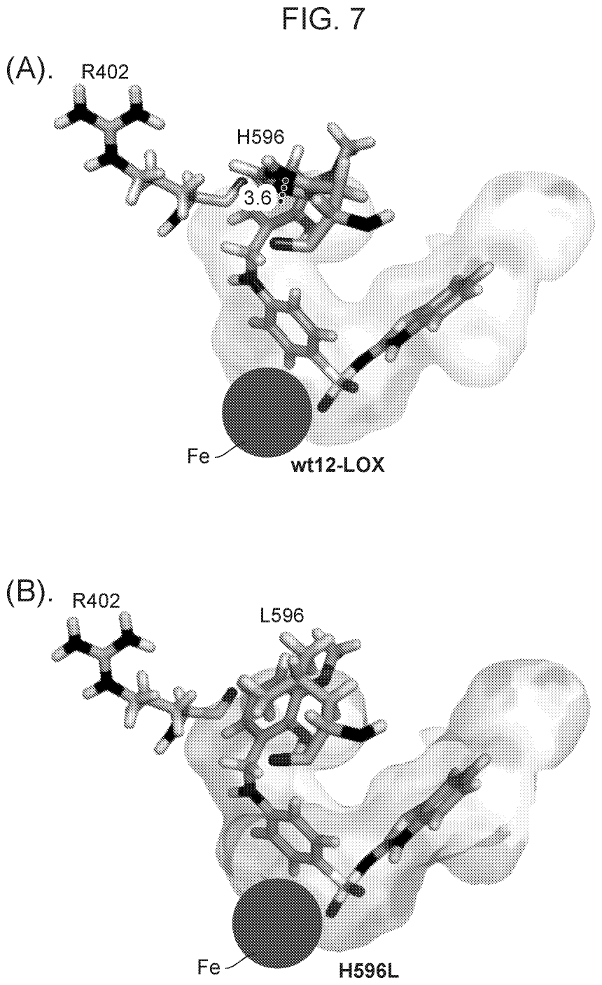

Given the improved potency of compound LOX-12-001 against h12-LOX, its potency in islet cells was investigated. Glucose-stimulated insulin secretion (GSIS) of normal human and mouse islet cells, demonstrate increased insulin production, however, when exposed to pro-inflammatory cytokines (PIC), the difference between stimulated and unstimulated cells decreases. This exposure of PIC to both human and mouse β cells upregulates the h12-LOX pathway, and the inhibition of this h12-LOX pathway was implicated in protecting the viability and function of human and mouse β cells after PIC exposure. Since ML355 was previously shown to be a highly selective inhibitor of h12-LOX with a favorable ADME profile in protecting human islets against the impairment of GSIS by PIC, compound LOX-12-001 was also tested (FIG. 8). As a control, human islet cells treated with 18 mmol/L of glucose (HG) showed a marked increase in insulin secretion (224+/−50 mU/L) relative to 3 mmol/L of glucose (LG) (24+/−4 mU/L). PIC treatment for 24 hrs increased basal insulin secretion at 3 mmol/L of glucose (189+/−54 mU/L) and led to the reduction of further increases in insulin secretion at 18 mmol/L of glucose (241.+/−50 mU/L). When 1 μM compound LOX-12-001 was added along with the PIC (for 24 hrs), the difference in insulin production was restored, 140+/−3 mU/L and 352+/−10 mU/L, respectively. Only compound LOX-12-001 (1 μM) was added as an additional control and a similar insulin secretion increase to vehicle was observed, 25+/−5 mU/L and 128+/−13 mU/L, respectively. For comparison, 10 μM of ML355 was utilized in a similar GSIS experiment and comparable results were observed, however, caution should be given to such comparisons given the differences between the batches of human islets.

### CONCLUSIONS

From the ML355 docking pose in the h12-LOX active site, residues were mutated with the potential of affecting ML355 binding and determined the ML355 inhibitor potency against the mutants. These results were then compared to the calculated wt12-LOX/ML355 binding affinities and the relationship between their docking score and inhibitor potency demonstrated a positive correlation, lending confidence to the docking model of MHL355 bound to the h12-LOX active site (FIG. 1).

The docking model predicts a number of interactions between the active site and ML355. A key characteristic of the h12-LOX active site is its curved nature, which has been described as a “U” shape. L407 defines the “U” shape of the h12-LOX active site and has been shown to properly position the fatty acid, AA, for catalysis. Appropriately, ML355 conforms to the “U” shape of the h12-LOX active site, having specific interactions with active site residues. L407 is at the bottom of the “U”, with ML355 wrapping around the residue, with interaction to the phenyl linker region of ML355. Reducing the size of L407 progressively decreases ML355 potency, manifesting a 7-fold decrease in the IC₅₀ value with L407G (FIG. 3), supporting the hypothesis that ML355 gains binding affinity from the curvature of the active site.

Beyond L407, the benzothiazole of ML355 extends into the bottom of the active site cavity, pointing towards the Sloane determinants, residues A417 and V418. However, the docking model indicates limited interaction between ML355 and these residues. This lack of interaction was confirmed by the lack of effect by the cavity reducing mutation, A417I/V418M. This result indicated that a potential active site cavity was present such that the volume of ML355 could be increased by attaching substituents to the benzothiazole moiety and thus optimize van der Waals interactions. In order to test this hypothesis, a derivative of ML355 with a larger napthyl-benzothiazole, compound LOX-12-001 (Scheme 1), was designed and shown to dock to the active site better than ML355. It was therefore synthesized and found to have 7.2-fold greater potency than ML355 against 12-LOX, supporting our docking model. Unexpectedly, the double mutant, A417I/V418M, only had a 2.4-fold increase in the IC₅₀ with compound LOX-12-001, suggesting additional cavity space even with compound LOX-12-001. If, however, the cavity volume was also narrowed, A417I/V418M/S594T, the IC₅₀ increased dramatically to greater than 20 uM, for both ML355 and compound LOX-12-001. This result suggests that both the depth and width of the active site affect inhibitor binding. The specificity of the inhibitor/active site interaction was also confirmed with loss of inhibitor potency with the triple mutant, L407G/A417I/V418M. The curious aspect of this mutation is that by increasing the size of the active site in the middle of the cavity, but reducing the size at the end of the cavity abolishes inhibitor potency, suggesting that both ML355 and compound LOX-12-001 interact with L407 in such a way as optimize their active site binding.

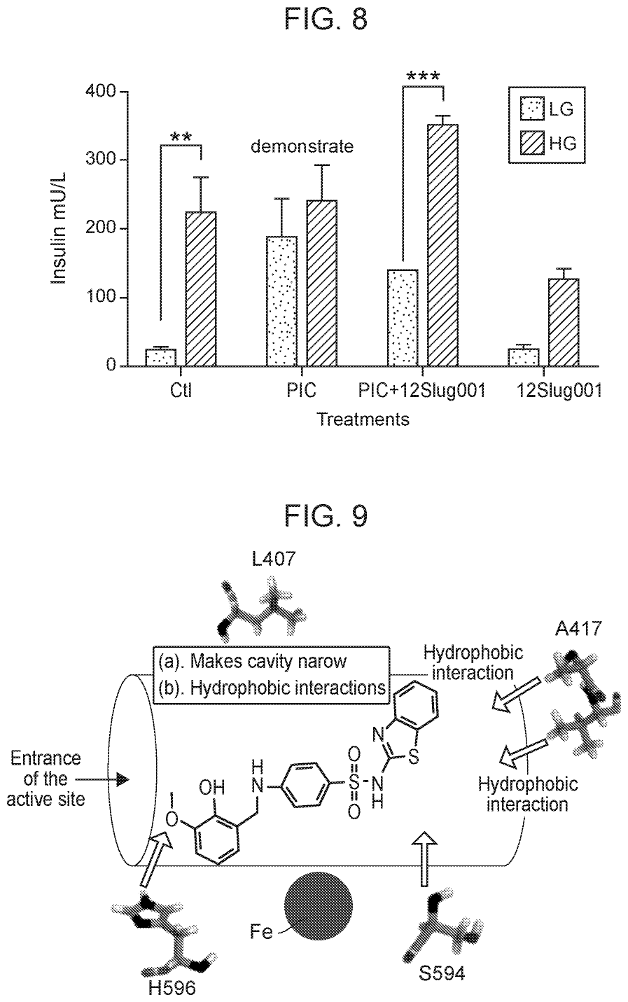

Another aspect of the active site of h12-LOX is the aromaticity on key residues, F414 and F352, which the model suggests interacts with the aromatic moieties of the inhibitors. However, loss of the aromaticity of F414 and F352 affected the potency of compound LOX-12-001 more so than ML355. This suggests a difference in their binding mode, with compound LOX-12-001 potentially having more pi-pi interactions with F414 and F352 than ML355. This is a reasonable hypothesis since compound LOX-12-001 has a more extensive pi-system than ML355. (FIG. 9)

Computational prediction and subsequent experimental study showed that H596 could play a role in anchoring the carboxylate of AA during catalysis but not R402. Therefore, both H596 and R402 were replaced with a nonpolar leucine and the inhibitory potency against ML355 was tested. H596L showed a greater increase in the IC₅₀ than that of R402 relative to wt12-LOX (5-fold increase for H596L and 0.8-fold for R402L), suggesting that H596 in h12-LOX may contribute to properly positioning ML355 binding in the active site but not R402, possibly through the interaction with the p-methoxy catechol moiety of ML355.

The gain in potency of compound LOX-12-001 supported the binding hypothesis however, its improved potency also suggested improved drug qualities. Its selectivity against other LOX isozymes was maintained and it maintained its potency in PIC challenged human islet cells, comparable to that of ML355 (FIG. 5). These results demonstrate that compound LOX-12-001 is more potent and efficacious than ML355.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a feature in the claim only when the exact phrase “means for” or the exact phrase “step for” is recited at the beginning of such feature in the claim; if such exact phrase is not used in a feature in the claim, then 35 U.S.C. § 112(f) or 35 U.S.C. § 112(6) is not invoked.

What is claimed is:

1. A compound of formula I: 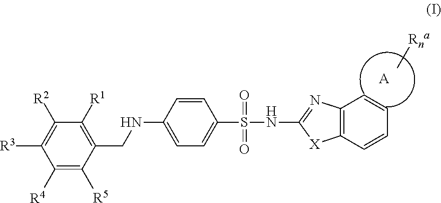
wherein R¹, R², R³, R⁴ and R⁵ are each independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl;
X is S or O;
the A ring is a substituted or unsubstituted 5 to 12 membered ring;
n is an integer from 0 to 12; and
each R^{a} is independently selected from hydrogen, hydroxy, alkoxy, amine, cyano, thiol, halogen, alkyl, substituted alkyl, heteroalkyl, substituted heteroalkyl, cycloalkyl, substituted cycloalkyl, heterocycloalkyl, substituted heterocycloalkyl, aryl, substituted aryl, arylalkyl, substituted arylalkyl, heteroaryl, substituted heteroaryl, heteroarylalkyl, and substituted heteroarylalkyl,
or a salt, solvate or hydrate thereof.

2. The compound according to claim 1, wherein:
R¹ is hydroxy;
R² is alkoxy; and
each of R³, R⁴ and R⁵ are hydrogen.

3. The compound according to any one of claims 1-2, wherein X is S.

4. The compound according to any one of claims 1-3, wherein the compound is of formula Ia: 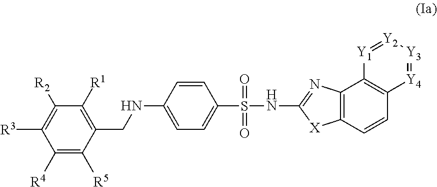
wherein Y₁, Y₂, Y₃ and Y₄ are each independently C or N.

5. The compound according to claim 9, wherein:
Y₁, Y₂, Y₃ and Y₄ are each C; or
Y₃ is N; and Y₁, Y₂, and Y₄ are each C; or
Y₁ and Y₄ are N and Y₂ and Y₃ are C.

6. The compound according to any one of claims 1-5, wherein:
A) A is unsubstituted; or
B) n is 1 or 2 and each R^{a} is independently selected from: 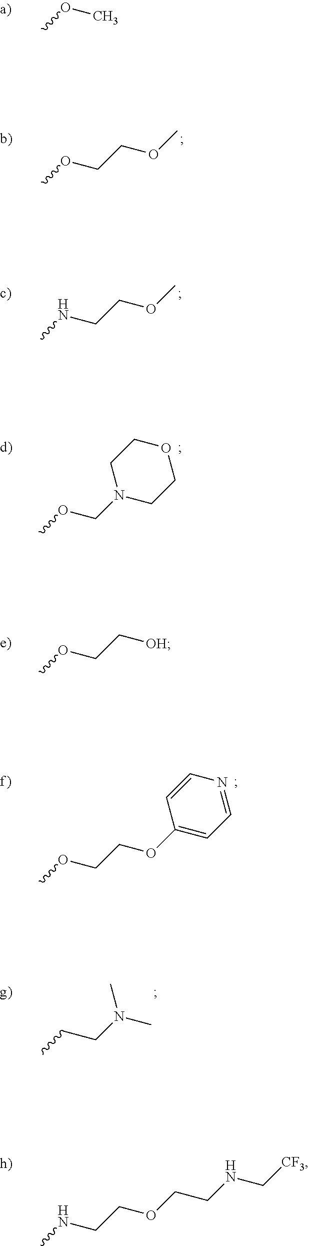
wherein [symbol] represents the A-R^{a} bond.

7. The compound according to any one of claims 4-6, wherein the compound is selected from:

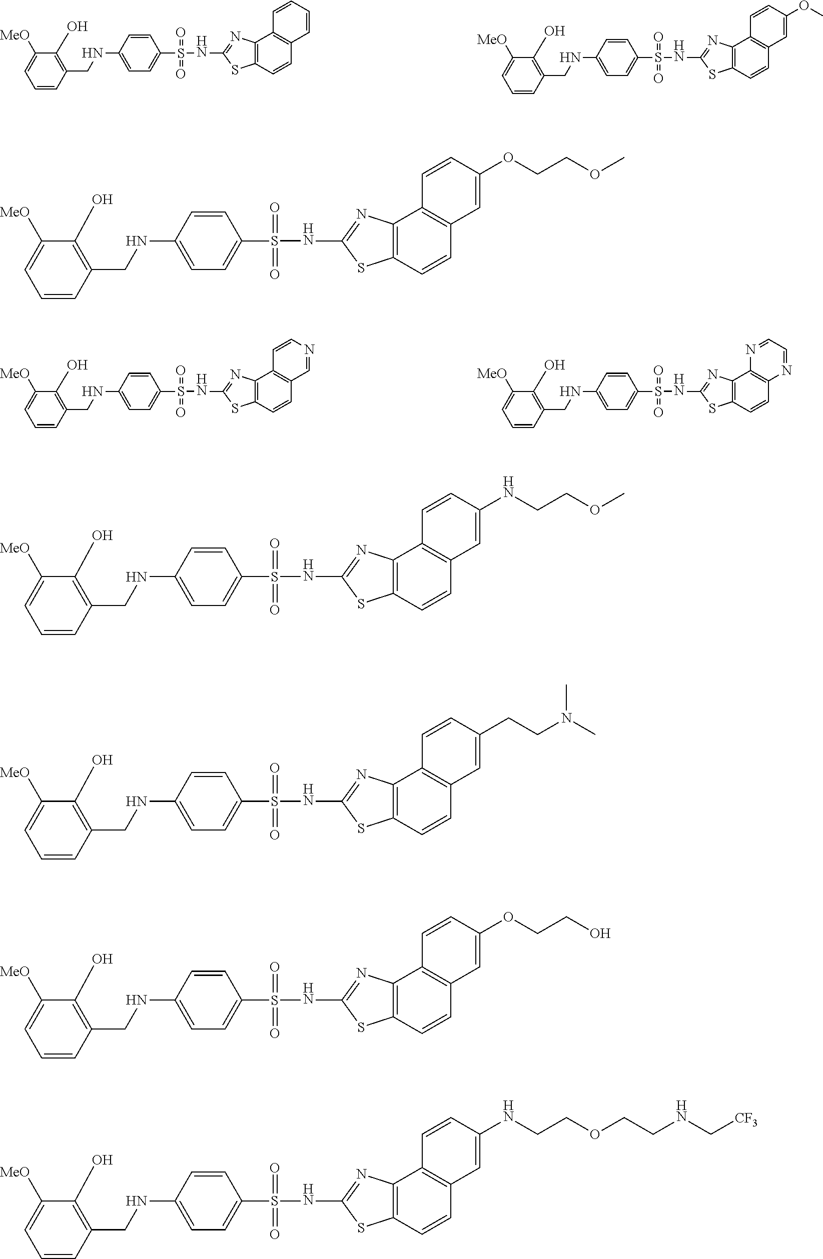

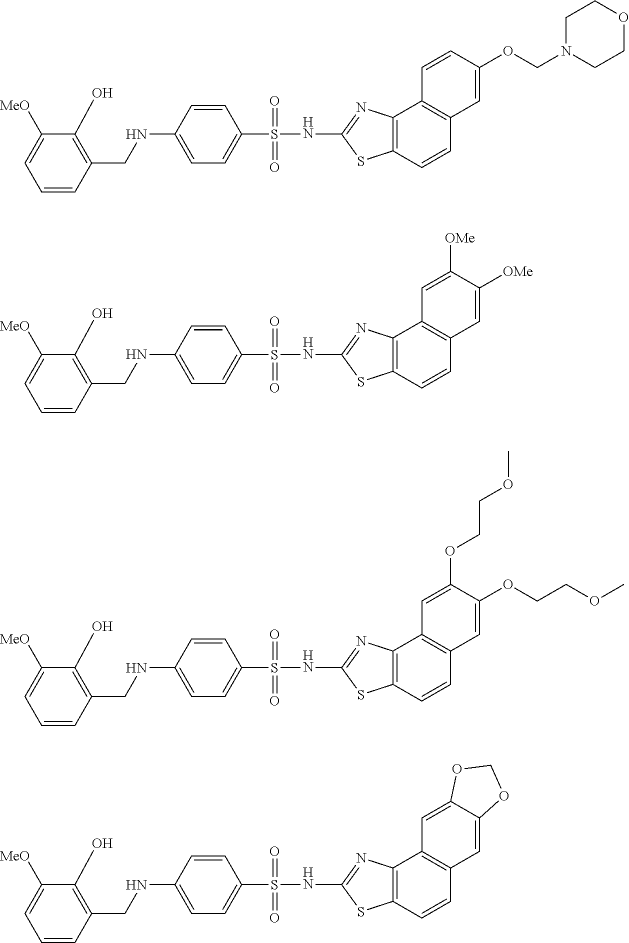 [SMILES: COc1cccc(CNc2ccc(S(=O)(=O)Nc3nc4c(ccc5cc(OCN6CCOCC6)ccc54)s3)cc2)c1O ; COc1cc2ccc3sc(NS(=O)(=O)c4ccc(NCc5cccc(OC)c5O)cc4)nc3c2cc1OC ; COCCOc1cc2ccc3sc(NS(=O)(=O)c4ccc(NCc5cccc(OC)c5O)cc4)nc3c2cc1OCCOC ; COc1cccc(CNc2ccc(S(=O)(=O)Nc3nc4c(ccc5cc6c(cc54)OCO6)s3)cc2)c1O]

8. The compound according to any one of claims 1-3, wherein the compound is of formula Ib: 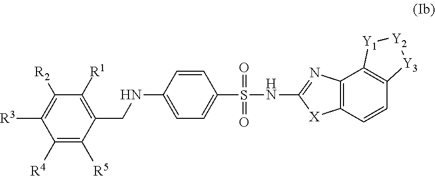
wherein Y₁, Y₂, Y₃ and Y₄ are each independently C, N or O.

9. The compound according to claim 19, wherein:
Y₁, Y₂ and Y₃ are each C; or.
Y₂ is C and Y₁ and Y₃ are O; or
Y₁ is N and Y₂ and Y₃ are C.

10. The compound according to any one of claims 8-9, wherein:
A) A is unsubstituted; or
B) n is 1 or 2 and each R^{a} is independently selected from: 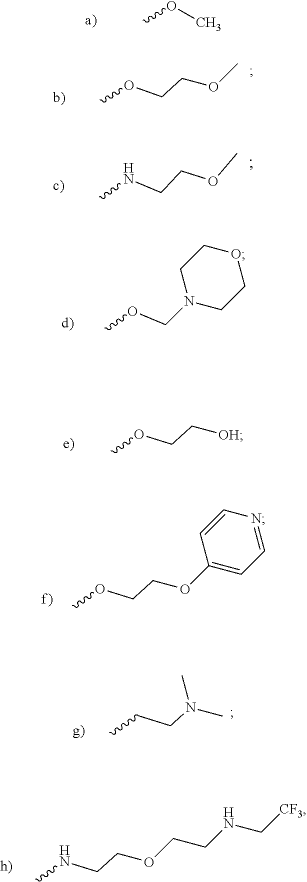
wherein [symbol] represents the A-R^{a} bond.

11. The compound according to any one of claims 8-10, wherein the compound is selected from:

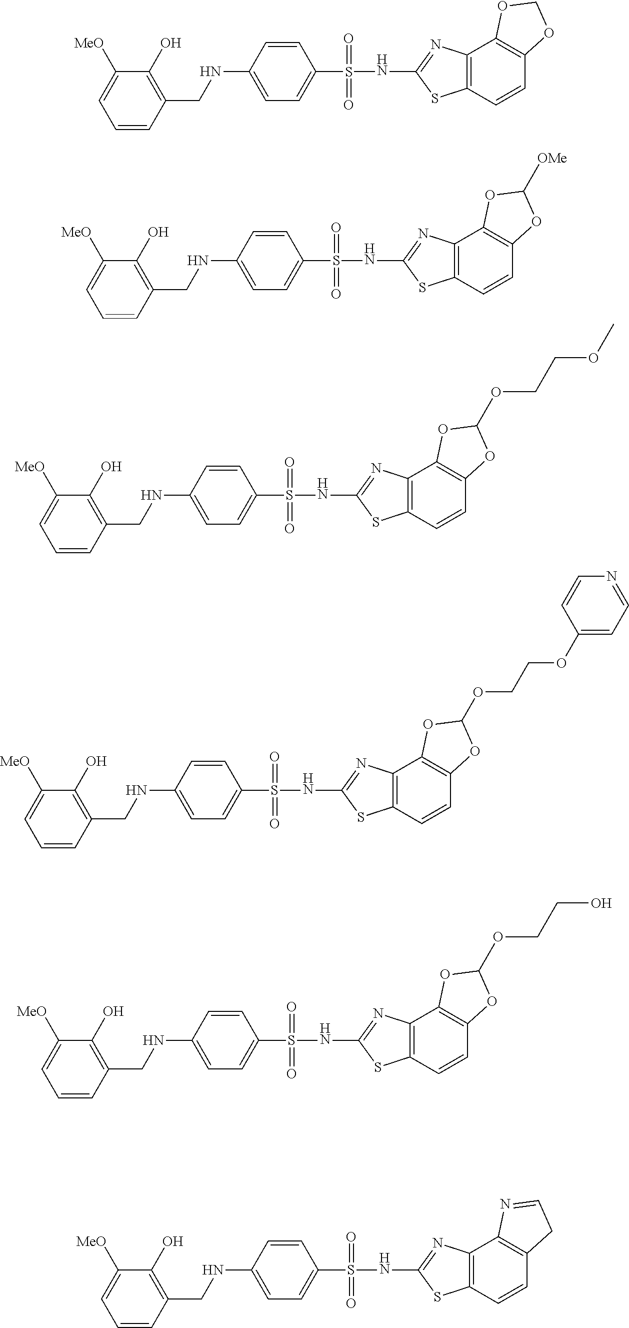 [SMILES: COc1cccc(CNc2ccc(S(=O)(=O)Nc3nc4c5c(ccc4s3)OCO5)cc2)c1O ; COc1cccc(CNc2ccc(S(=O)(=O)Nc3nc4c5c(ccc4s3)OC(OC)O5)cc2)c1O ; COCCOC1Oc2ccc3sc(NS(=O)(=O)c4ccc(NCc5cccc(OC)c5O)cc4)nc3c2O1 ; COc1cccc(CNc2ccc(S(=O)(=O)Nc3nc4c5c(ccc4s3)OC(OCCOc3ccncc3)O5)cc2)c1O ; COc1cccc(CNc2ccc(S(=O)(=O)Nc3nc4c5c(ccc4s3)OC(OCCO)O5)cc2)c1O ; COc1cccc(CNc2ccc(S(=O)(=O)Nc3nc4c5c(ccc4s3)CC=N5)cc2)c1O]

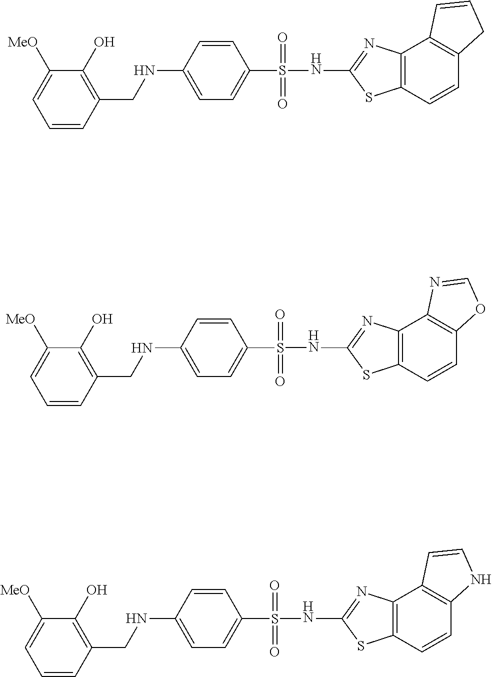 [SMILES: COc1cccc(CNc2ccc(S(=O)(=O)Nc3nc4c5c(ccc4s3)CC=C5)cc2)c1O ; COc1cccc(CNc2ccc(S(=O)(=O)Nc3nc4c(ccc5ocnc54)s3)cc2)c1O ; COc1cccc(CNc2ccc(S(=O)(=O)Nc3nc4c(ccc5[nH]ccc54)s3)cc2)c1O]

12. 4-((2-hydroxy-3-methoxybenzyl)amino)-N-(naphtho[1,2-d]thiazol-2-yl)benzenesulfonamide: 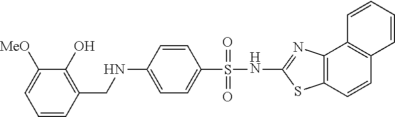 [SMILES: COc1cccc(CNc2ccc(S(=O)(=O)Nc3nc4c(ccc5ccccc54)s3)cc2)c1O]
or a pharmaceutically acceptable salt, solvate or hydrate thereof.

13. A composition comprising:
a compound according to any one of claims 1-12; and
a pharmaceutically acceptable excipient.

14. A method for inhibiting human platelet 12-(S)-lipoxygenase, the method comprising contacting a cell with a compound according to any one of claims 1-12 or a composition of claim 13.

15. A method comprising administering to a subject in need thereof a therapeutically effective amount of a compound according to any one of claims 1-12 or a composition of claim 13.
